(19) United States
(12) Patent Application Publication
(10) Pub. No.: US 2014/0127814 A1
(43) Pub. Date: May 8, 2014

(54) GENERATION AND USE OF PLURIPOTENT STEM CELLS

(71) Applicants: Srinivasan Chandrasegaran, Baltimore, MD (US); Ramalingam Sivaprakash, Baltimore, MD (US); Karthikeyan Kandavelou, Puducherry (IN); Viktoriya London, Rockville, MD (US)

(72) Inventors: Srinivasan Chandrasegaran, Baltimore, MD (US); Ramalingam Sivaprakash, Baltimore, MD (US); Karthikeyan Kandavelou, Puducherry (IN); Viktoriya London, Rockville, MD (US)

(21) Appl. No.: 13/816,483

(22) PCT Filed: Aug. 10, 2011
(86) PCT No.: PCT/US2011/047268

§ 371 Date: Aug. 9, 2013

Related U.S. Application Data
(60) Provisional application No. 61/372,166, filed on Aug. 10, 2010.

(51) Int. Cl. C12N 15/90

(52) U.S. Cl. CPC C12N 15/907
USPC 435/462

(57) ABSTRACT

ABSTRACT Methods, compositions, constructs, vectors, cell lines, and kits, for generating induced pluripotent stem cells by site-specific integration of pluripotency coding sequences with endonucleases for use in gene therapy, regenerative medicine, cell therapy or drug screening.

The research resulting in the invention described herein was supported in part by funding from the National Institutes of Health GM077291. The United States Government has certain rights in the invention.

### BACKGROUND

Custom zinc finger nucleases (ZFNs) have become powerful tools to deliver a targeted double strand break at a pre-determined chromosomal locus within the human genome and induce homology-directed gene insertion in the presence of exogenously provided donor DNA carrying the transgenes. The creation of designed zinc finger nucleases (ZFNs), and the development of ZFN-mediated gene targeting, has provided molecular biologists with the ability to site-specifically and permanently modify plant and mammalian genomes, including the human genome via homology-directed repair of a targeted genomic DSB (8-12). ZFN-mediated gene modification has been successfully demonstrated in a variety of human cells and cell types (13-28). High rate of endogenous gene modification efficiencies (>10%) have been achieved using this approach (14).

A potential limitation of the ZFN-mediated targeting approach is off-target DNA cleavage at related sequences (like the CCR2 locus when targeting CCR5), which may cause unpredictable genotoxic effects (10). Generation of human induced pluripotent stem cells (hiPSCs) using site-specific integration with phage integrase (ΦC31) has been reported elsewhere, for example reference (29). Although the derived hiPSCs had only a single integration in each line, the locations of integration were random in different lines, favoring intergenic regions. Genetic engineering of hiPSCs using designed ZFNs or TALE nucleases (TALENs) have been reported previously in literature, for example references (20-25); however, the hiPSCs used in these studies were generated by using standard viral vector reprogramming methods, which employs random integrations of pluripotency genes within the human genome (30).

There is a need for a more efficient method for generating hiPSCs using more precise and deliberate site-specific integration and insertion at safe harbor locus of the human genome avoiding critical genes or control regions, for functional complementation studies and to treat monogenic diseases.

### BRIEF DESCRIPTION OF THE DRAWINGS

FIG. 1. Schematic diagram showing efficient generation of hiPSCs by targeted insertion of stem cell factor genes at the CCR5 locus of the human lung fibroblasts and reshaping the functionality of heterozygous hiPSCs after Cre recombinase treatment by targeted addition and expression of wild-type CFTR transcription unit at the wild type CCR5 allele, using designed ZFNs.

FIG. 2. Efficient generation of hiPSCs from human fibroblasts by targeted insertion of Oct4/Sox2 transcription factors at the CCR5 locus using designed ZFNs, in conjunction with small molecule inhibitor, VPA. (I) Morphology of precisely targeted CCR5-disrupted hiPSCs before CRE treatment. Panels: (A & B), Bright field images of the morphology two individual CCR5-modified hiPSC colonies generated by co-transfection of human fibroblasts (IMR90 cells) with CCR5-specific ZFNs and donor constructs; (C & D), GFP fluorescence images of hiPSC single cell colonies shown in A & B; (E & F), Alkaline phosphatase staining of two CCR5-modified hiPSC single cell colonies is shown. Immuno-staining for Oct4/Sox2/Nanog/Tra1-60 and DAPI staining of hiPSCs are also shown below. (II) Morphology of precisely targeted CCR5-disrupted hiPSCs after CRE treatment. Panels: (A), Bright field images of the morphology three CCR5-modified hiPSC single cell colonies generated by co-transfection of human fibroblasts (IMR90 cells) with CCR5-specific ZFNs and donor constructs; (B), GFP fluorescence images of hiPSC single cell colonies shown in A. Immuno-staining for Oct4/Sox2/Nanog/Tra1-60 and DAPI staining of hiPSCs are also shown below. (III) Schematic representation of donor (Oct4/Sox2/eGFP flanked by CCR5 homology arms) insertion site at the CCR5 locus of heterozygous hiPSCs. Endogenous genomic primers outside the CCR5 homology arms and anchor primers inside the donor for 5′ and 3′ junction site, respectively, are also shown. (IV) PCR analysis of 5′ junction of donor insertion site in four different CCR5-disrupted hiPSC single cell colonies, before CRE treatment is shown. Lanes: 1-4, single allele CCR5-disrupted hiPSC colonies; 5, Control IMR90 cells; and 6, 1 Kb ladder. PCR analysis yields the expected band size (1.8 kb) confirming insertion of the donor at the CCR5 locus. (V) PCR analysis of 3′ junction of donor insertion site in four different CCR5-disrupted hiPSC single cell colonies, before CRE treatment is shown. Lanes: 1-4, single allele CCR5-disrupted hiPSC colonies; 5, Control IMR90 cells; and 6, 1 Kb ladder. PCR analysis yields the expected band size (2.7 kb) confirming insertion of the donor at the CCR5 locus.

FIG. 3. Characterization of CCR5-disrupted bi allele mutant hiPSCs, generated from human fibroblasts by targeted insertion of Oct4/Sox2 transcription factors at the CCR5 locus using designed ZFNs, in conjunction with small molecule inhibitor, VPA. (I) Morphology of precisely targeted CCR5-biallele-modified hiPSCs before CRE treatment. Panels: (A & B), Bright field images of the morphology two individual CCR5-modified hiPSC colonies generated by co-transfection of human fibroblasts (IMR90 cells) with CCR5-specific ZFNs and donor constructs; (C & D), GFP fluorescence images of hiPSC single cell colonies shown in A & B; (E & F), Alkaline phosphatase staining of two CCR5-modified hiPSC single cell colonies is shown. Immuno-staining for Oct4/Sox2/Nanog/Tra1-60 and DAPI staining of hiPSCs are also shown below. (II) Morphology of precisely targeted CCR5-disrupted hiPSCs after CRE treatment. Panels: (A), Bright field images of the morphology three CCR5-modified hiPSC single cell colonies generated by co-transfection of human fibroblasts (IMR90 cells) with CCR5-specific ZFNs and donor constructs; (B), GFP fluorescence images of hiPSC single cell colonies shown in A. Immuno-staining for Oct4/Sox2/Nanog/Tra1-60 and DAPI staining of hiPSCs are also shown below. (III) Schematic representation of donor (Oct4/Sox2/eGFP flanked by CCR5 homology arms) insertion site at the CCR5 locus of heterozygous hiPSCs. Endogenous genomic primers outside the CCR5 homology arms and anchor primers inside the donor for 5′ and 3′ junction site, respectively, are also shown. (IV) PCR analysis of 5′ junction of donor insertion site in four different CCR5-disrupted hiPSC single cell colonies, before CRE treatment is shown. Lanes: 1-4, single allele CCR5-disrupted hiPSC colonies; 5, Control IMR90 cells; and 6, 1 Kb ladder. PCR analysis yields the expected band size (1.8 kb) confirming insertion of the donor at the CCR5 locus. (V) PCR analysis of 3′ junction of donor insertion site in four different CCR5-disrupted hiPSC single cell colonies, before CRE treatment is shown. Lanes: 1-4, single allele CCR5-disrupted hiPSC colonies; 5, Control IMR90 cells; and 6, 1 Kb ladder. PCR analysis yields the expected band size (2.7 kb) confirming insertion of the donor at the CCR5 locus.

FIG. 4. A: Sequence analysis of 5′ junction of the donor insertion sites of heterozygous CCR5-mutant hiPSCs and biallele mutant hiPSCs, before CRE treatment (See also Table 1A); B: Sequence analysis of 3′ junction of the donor insertion sites in heterozygous CCR5-mutant hiPSCs and biallele mutant hiPSCs, before CRE treatment (See also Table 1B); C: Analysis of the CCR5 locus repaired by NHEJ in the biallele mutant hiPSCs, before CRE treatment (See also Table 1C).

FIG. 5. PCR and nucleotide sequence analysis of the mutated CCR5 locus in single cell colonies of heterozygous (single allele mutant) hiPSCs and biallele mutant hiPSCs. (I) Schematic diagram showing donor insertions sites at the CCR5 locus in heterozygous hiPSCs and biallele mutant hiPSCs before and after CRE treatment. (II) PCR amplification of the mutant CCR5 locus using genomic DNA from single cell colonies of heterozygous hiPSCs and primers flanking the CCR5-specific ZFN target sites. (III) PCR amplification of the mutant CCR5 locus using genomic DNA from single cell colonies of biallele mutant hiPSCs and primers flanking the mutation site(s). The PCR fragments were then cloned and sequenced to determine the nucleotide sequence at the mutated CCR5 locus.

FIG. 6. A: Sequence analysis of the CCR5 mutant allele in heterozygous hiPSCs, after CRE treatment (See also Table 2A); B: Sequence analysis of the CCR5 mutant alleles in the bi-allele mutant hiPSCs, after CRE treatment (See also Table 2B).

FIG. 7. (A) ZFN-mediated targeted insertion of the WT CFTR transcription unit at the transduced CCR5 locus in HEK293 Flp-In cells. (i), CFTR donor plasmid; (ii), Flow cytometry analysis of CCR5 expressing Flp-In cells 1-week post-transfection of 4-finger CCR5 ZFNs (A), Isotype control; (B), Positive control (no ZFNs transfection). Cells were stained with Mab against hCCR5. CCR5+ cells (>93%) are quantified in region (+). (C) Cells 1-week post-transfection with 4-finger ZFNs and CFTR donor plasmid: 23% cells became CCR5⁻, which is quantified in region (−). CCR5-gene modified single cells were isolated by FACS, grown and CFTR expression analyzed. (iii), Western blot profile of two representative CCR5-modified single cell clones for CFTR expression using CFTR antibody (lanes 1 & 2); lane 3, intentionally left blank; and lane 4, T84 cell line. (B) ZFN-mediated targeted insertion of the CFTRΔ508F transcription unit at the transduced CCR5 locus in HEK293 Flp-In cells. (i), CFTRΔ508F donor plasmid; (ii), Flow cytometry analysis of CCR5 expressing Flp-In cells 1-week post-transfection 4-finger CCR5 ZFNs (A), Isotype control; (B), Positive control (no ZFNs transfection). Cells were stained with mAB against hCCR5. CCR5+ cells (>93%) are quantified in region (+). (C) Cells 1-week post-transfection with 4-finger ZFNs and CFTRΔ508F donor: 17% cells became CCR5 negative, which is quantified in region (R8). CCR5-gene modified single cells were isolated by FACS, grown and CFTRΔ508F expression analyzed. (iii), Western blot profile of a CCR5-modified single cell clone for CFTRΔ508F expression using CFTR antibody. Lanes: 1, WT_CFTR clone (Scl); 2, CFTRΔ508F clone; and 3, Negative control.

FIG. 8. Analysis of the endogenous CCR5 locus of HEK293 cell lines expressing the WT_CFTR and mutant CFTRΔ508F protein, respectively. (See also Table 3).

FIG. 9. Targeted addition and expression of wild-type CFTR transcription unit from the wild type CCR5 allele, of the heterozygous hiPSCs using designed ZFNs. (i) Morphology of heterozygous hiPSCs with precisely targeted addition of the CFTR transcription unit at the wild type CCR5 allele. Panels: A) Bright field images of the morphology of three single cell CFTR hiPSC colonies; (B) tdtomato fluorescence images of CFTR hiPSC colonies shown in A. Immuno-staining for Oct4/Sox2/Nanog and DAPI staining of three different CFTR hiPSCs are also shown. (II) Schematic representation of donor (tdtomato/CFTR flanked by CCR5 homology arms) insertion site at the CCR5 locus of heterozygous hiPSCs. Endogenous genomic primers outside the CCR5 homology arms and anchor primers inside the donor for both 5′ and 3′ junction sites, respectively, are also shown. (III) PCR analysis of 5′ junction of donor insertion site in five different CCR5-disrupted CFTR hiPSC single cell colonies is shown. Lanes: 1-5, CCR5-disrupted CFTR hiPSC colonies; 5, 1 Kb ladder. PCR analysis yields the expected band size (1.8 kb) confirming insertion of the donor at the remaining CCR5 wild type allele. (IV) PCR analysis of 3′ junction of donor insertion site in five different CCR5-disrupted CFTR hiPSC single cell colonies is shown. Lanes: 1-5, CCR5-disrupted CFTR hiPSC colonies; 5, 1 Kb ladder. PCR analysis yields the expected band size (1.4 kb) confirming insertion of the donor at the remaining CCR5 wild type allele in heterozygous hiPSCs. (V) The Western blot was profile of four different CCR5-disrupted CFTR hiPSCs, probed using the antibody Ab217, anti C-terminal monoclonal mouse antibody against CFTR (purchased from UNC Center for Cystic Fibrosis Research) is shown. Lanes: 1, IMR90 cells; 2, control heterozygous hiPSCs before tdtomato/CFTR insertion at the remaining CCR5 wild type allele; and 3 to 6, four different single cell colonies of CCR5-disrupted CFTR hiPSCs. Efficient expression of the fully glycosylated CFTR from the CCR5 locus is observed in all the CFTR hiPSCs that we examined as seem from the prominent C-band.

FIG. 10. A: Sequence analysis of 5′ junction of the tdTomato/CFTR donor insertion site in heterozygous CCR5-mutant hiPSCs (See also Table 4A); B: Sequence analysis of 3′ junction of the tdTomato/CFTR donor insertion site in heterozygous CCR5-mutant hiPSCs (See also Table 4B).

FIG. 11. PCR primer sequences and amplification conditions (See also Table 5).

FIG. 12. Targeted addition and ectopic expression of tdTomato/β-globin gene from the CCR5 locus of TNC1 hiPSC cell line, which contains homozygous Sickle Cell Disease mutation (SCD) for functional complementation. TNC1 line (Chou et al., 2011) was purchased from Dr. Linzhao Cheng lab (Johns Hopkins School of Medicine, Baltimore, USA). The frozen TNC1 hiPScs were thawed and cultured under MEF feeder condition. For nucleofection, TNC1 hiPSCs were passaged onto 6-well matrigel plates and cultured in mTesRI (Stem cell Technologies) under feeder-free conditions. Two million TNC1 hiPSCs were digested with accutase (Sigma) for 2-3 min and neutralized by PBS. The cells were centrifuged at 100×g for 5 min, and resuspended in 100 μl of Amaxa nucleofector solution V (Amaxa Biosystems Gaithersburg Md.) with 1 μg of each pIRES vector carrying the corresponding CCR5-specific ZFNs and 8 μg of the donor plasmid containing the wild-type beta-globin and marker gene tdTomato. The whole transgene cassette was flanked by 750 bp endogeneous CCR5 locus-specific sequence on both the sides for ZFN-evoked homology directed repair. The TNC1 hiPSCs were transfected using an Amaxa nuclofector device with program A-023. 10 μM ROCK inhibitor Y 27632 was added for 1 hr prior to and immediately after nucleofection to improve the survival of dissociated hiPSCs. After nucleofection, the cells were seeded in matrigel plates and allowed to grow for at least a week before cell sorting. A) tdTomato fluorescence images of CCR5-modified SCD hiPSCs; B) Bright field images of CCR5-modified SCD hiPSCs. The tdTomato-expressing cells will be sorted by FACS. Serial dilution will be used to isolate single cell colonies, which will be characterized by PCR, sequencing and immunostaining as well as monitored for β-globin gene expression.

### DESCRIPTION

Disclosed are methods and compositions for generating stem cells from a target cell, such as a somatic cell or primary cell.

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolfe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolfe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

In some embodiments, the target cell is a somatic cell or primary cell.

Somatic cells for use with the present invention may be primary cells or immortalized cells. Such cells may be primary cells (non-immortalized cells), such as those freshly isolated from an animal, or may be derived from a cell line (immortalized cells). In an exemplary aspect, the somatic cells are mammalian cells, such as, for example, human cells or mouse cells. They may be obtained by well-known methods, from different organs, such as, but not limited to skin, lung, pancreas, liver, stomach, intestine, heart, reproductive organs, bladder, kidney, urethra and other urinary organs, or generally from any organ or tissue containing living somatic cells. Mammalian somatic cells useful in the present invention include, by way of example, adult stem cells, sertoli cells, endothelial cells, granulosa epithelial cells, neurons, pancreatic islet cells, epidermal cells, epithelial cells, hepatocytes, hair follicle cells, keratinocytes, hematopoietic cells, melanocytes, chondrocytes, lymphocytes (B and T lymphocytes), erythrocytes, macrophages, monocytes, mononuclear cells, fibroblasts, cardiac muscle cells, other known muscle cells, and generally any live somatic cells. In particular embodiments, fibroblasts are used. The term somatic cell, as used herein, is also intended to include adult stem cells. An adult stem cell is a cell that is capable of giving rise to all cell types of a particular tissue. Exemplary adult stem cells include hematopoietic stem cells, neural stem cells, and mesenchymal stem cells.

In particular embodiments, the target cell is human lung fibroblasts.

In other embodiments, the stem cell may be pluripotent cells or an induced pluripotent stem cell.

As used herein, stem cells or pluripotent cells include cells that have the potential to divide in vitro for an extended period of time (greater than one year) and have the unique ability to differentiate into cells derived from all three embryonic germ layers, including the endoderm, mesoderm and ectoderm.

In particular embodiments, the stem cell, the induced pluripotent stem cell, the somatic cell and primary cell are human. In some aspects, the stem cell may be adult human stem cells, human stem progenitor cells (hSPC) or human induced pluripotent stem cells (hiPSC).

In other aspects, the induced pluripotent stem cell, the somatic cell and the primary cell are non-human. In some aspects, the stem cell may be non-human embryonic stem cells, non-human adult stem cells, non-human stem progenitor cells or non-human induced pluripotent stem cells.

In particular embodiments the methods include introducing into the target cell one or more of pluripotency coding sequences at a safe-harbor locus within the cell target genome using site-specific endonucleases, the one or more of a pluripotency coding sequences giving rise upon transcription to a factor that contributes to the reprogramming of said target cell into an induced pluripotent stem cell.

The term “introducing” as used in accordance with the present invention relates to the process of bringing the coding sequences into the target cell and subsequently incorporation of said coding sequences into a safe harbor locus of the genomic DNA of the target cell using zinc finger nucleases (ZFNs) or transcription activator-like effector nucleases (TALENs). As used herein, this process is referred to as stable site-specific transfection, as stable transfection may include random transfection in a cell. Methods for stable transfection are well-known to the person skilled in the art and described, e.g., in Bonetta, L., (2005), Nature Methods 2, 875-883. Due to the low rate of reprogramming events taking place in transfected cells it is advantageous to rely on an efficient stable transfection method. Hence, the coding sequences are preferably introduced into a target cell by a method achieving high transfection/infection efficiency. For example, transfection/infection efficiencies of at least 30%, at least 50%, or at least 80% are preferred.

Suitable methods of transfection include, for example, lipofection, electroporation, nucleofection, magnetofection or viral vector infection. Preferably, retroviral vectors are used to achieve transfection of the target cells as said vectors not only mediate efficient entry of the coding sequences into the target cell but also their integration into the genomic DNA of the target cell. Retroviral vectors have shown to be able to transduce a wide range of cell types from different animal species, to integrate genetic material carried by the vector into target cells, to express the transduced coding sequences at high levels, and, advantageously, retroviral vectors do not spread or produce viral proteins after infection. Suitable retroviral vector systems are well-known to the person skilled in the art such as, e.g., retroviral vectors with the MoMuLV LTR, the MESV LTR, lentiviral vectors with various internal promoters like the CMV promoter, preferably with enhancer/promoter combinations that show silencing of transgene expression in embryonic/pluripotent cells. Episomal vector systems like adenovirus vectors, other non-integrating vectors, episomally replicating plasm ids could also be used. Preferably, the retroviral MX vector system is used in the method of the invention (Kitamura et al., (2003), Exp Hematol., 31(11):1007-1014).

Introducing, integrating, introduction or integration as used herein may be by way of recombination. “Recombination” refers to a process of exchange of genetic information between two polynucleotides. For the purposes of this disclosure, “homologous recombination (FIR)” refers to the specialized form of such exchange that takes place, for example, during repair of double-strand breaks in cells. This process requires nucleotide sequence homology, uses a “donor” molecule to template repair of a “target” molecule (i.e., the one that experienced the double-strand break), and is variously known as “non-crossover gene conversion” or “short tract gene conversion,” because it leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized FIR often results in an alteration of the sequence of the target molecule such that part or all of the sequence of the donor polynucleotide is incorporated into the target polynucleotide.

In some embodiments, there are two, three, four or five pluipotency genes inserted at the safe harbor locus, e.g., CCR5 locus or AAVS1 locus, to achieve reprogramming to hiPSC from somatic cell or primary cells.

As used herein, reprogramming is intended to refer to a process that alters or reverses the differentiation status of a somatic cell that is either partially or terminally differentiated.

Reprogramming of a somatic cell may be a partial or complete reversion of the differentiation status of the somatic cell. In an exemplary aspect, reprogramming is complete wherein a somatic cell is reprogrammed into an induced pluripotent stem cell. However, reprogramming may be partial, such as reversion into any less differentiated state. For example, reverting a terminally differentiated cell into a cell of a less differentiated state, such as a multipotent cell.

An “accessible region” is a site in cellular chromatin in which a target site present in the nucleic acid can be bound by an exogenous molecule which recognizes the target site. Without wishing to be bound by any particular theory, it is believed that an accessible region is one that is not packaged into a nucleosomal structure. The distinct structure of an accessible region can often be detected by its sensitivity to chemical and enzymatic probes, for example, nucleases.

A “target site” or “target sequence” is a nucleic acid sequence that defines a portion of a nucleic acid to which a binding molecule will bind, provided sufficient conditions for binding exist. For example, the sequence 5′-GAATTC-3′ is a target site for the Eco RI restriction endonuclease.

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

A “gene,” for the purposes of the present disclosure, includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of a mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

A “region of interest” is any region of cellular chromatin, such as, for example, a gene or a non-coding sequence within or adjacent to a gene, in which it is desirable to bind an exogenous molecule. Binding can be for the purposes of targeted DNA cleavage and/or targeted recombination. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

With respect to fusion polypeptides, the term “operatively linked” can refer to the fact that each of the components performs the same function in linkage to the other component as it would if it were not so linked. For example, with respect to a fusion polypeptide in which a ZFP or TALE DNA-binding domain is fused to a cleavage domain, the ZFP or the TALE DNA-binding domain and the cleavage domain are in operative linkage if, in the fusion polypeptide, the ZFP or TALE DNA-binding domain portion is able to bind its target site and/or its binding site, while the cleavage domain is able to cleave DNA in the vicinity of the target site.

Target cells to be used in the method of the invention can be derived from existing cells lines or obtained by various methods including, for example, obtaining tissue samples in order to establish a primary cell line. Methods to obtain samples from various tissues and methods to establish primary cell lines are well-known in the art (see e.g. Jones and Wise, Methods Mol Biol. 1997). Suitable somatic cell lines may also be purchased from a number of suppliers such as, for example, the American tissue culture collection (ATCC), the German Collection of Microorganisms and Cell Cultures (DSMZ) or PromoCell GmbH, Sickingenstr. 63/65, D-69126 Heidelberg.

In accordance with the method of the invention, a suitable target cell endogenously expresses factors selected from Oct3/4 or factors belonging to the Myc, Klf and Sox families of factors, wherein said factors in combination with exogenously introduced factors selected from the complementary set of factors, i.e. Oct3/4 or factors belonging to the Myc, Klf and Sox families of factors, are capable to reprogram a non-pluripotent target cell into an iPS cell. The cell resulting from the introduction of the one or two coding sequences expresses the combination of factor Oct3/4 and at least one factor of each family of factors selected from the group of Myc, Klf and Sox. The person skilled in the art is well-aware of methods to determine whether at least two of the above-described factors are endogenously expressed in a target cell.

The term “pluripotency coding sequence” relates to a nucleotide sequence that upon transcription gives rise to the encoded product. The transcription of the coding sequence in accordance with the present invention can readily be effected in connection with a suitable promoter. Preferably, the coding sequence corresponds to the cDNA sequence of a gene that gives rise upon transcription to a factor that contributes to the reprogramming of a target cell into an induced pluripotent stem cell, wherein the reprogramming factors in accordance with the method of the invention are selected from Oct3/4 or factors belonging to the Myc, Klf and Sox families of factors. In particular embodiments, it represents a gene, e.g., stem cell factor genes.

A “factor that contributes to the reprogramming of a target cell into an induced pluripotent stem cell” relates to a factor that is capable of contributing to the induction of the reprogramming of target cells into induced pluripotent stem cells, wherein the factor is selected from Oct3/4 and factors belonging to the Myc, Klf and Sox families of factors. Such reprogramming factors include, for example, Oct3/4, Sox2, Sox 1, Sox3, c-Myc, n-Myc, I-Myc, Klf1, Klf2, Klf4, Klf 5, and the like, or mutants thereof with retained reprogramming capabilities. Said contribution to the reprogramming may be in the form of, for example, changing the methylation pattern of a cell to one similar to an embryonic stem cell, shifting the expression profile of a cell towards the expression profile of an embryonic stem cell or affecting conformation of the aggregated nuclear DNA by modulating the histone binding similar to that observed in an embryonic stem cell wherein each of said changes may be effected either alone or in combination by a suitable reprogramming factor. Apart from the above-recited factors, the skilled person is aware of methods to identify further suitable reprogramming factors such as, e.g., bisulphite genomic sequencing, RT-PCR, real-time FOR, microarray analysis, karyotype analysis, teratoma formation, alkaline phosphatase staining, all of which are well-known to the person skilled in the art and are, for example described in Okita, K., et al. (2007), Nature 448(7151): 313-7; Park, I. H., et al. (2008), Nature 451(7175): 141-6; Takahashi, K., et al. (2007), Cell 131(5): 861-72; Wernig, M., et al. (2007), Nature 448(7151): 318-24; Takahashi, K. et al. (2007), Nat Protoc 2(12): 3081-9; or Hogan, B., et al. (1994), “Manipulating the Mouse Embryo: A Laboratory Manual”, Cold Spring Harbour Press.

Oct3/4 belongs to the family of octamer (“Oct”) transcription factors, and plays a role in maintaining pluripotency. The absence of Oct3/4 in cells normally expressing Oct3/4, such as blastomeres and embryonic stem cells, leads to spontaneous trophoblast differentiation. Thus, the presence of Oct3/4 contributes to the pluripotency and differentiation potential of embryonic stem cells. Various other genes in the “Oct” family, including Oct1 and Oct6, fail to elicit induction, thus demonstrating the exclusiveness of Oct3/4 to the induction process. The term “Oct4” is used herein interchangeably with the term “Oct3/4”.

The Sox family of genes is associated with maintaining pluripotency similar to Oct3/4, although it is associated with multipotent and unipotent stem cells in contrast to Oct3/4, which is exclusively expressed in pluripotent stem cells. Klf4 of the Klf family of genes was initially identified as a factor for the generation of mouse iPS cells and was demonstrated as a factor for generation of human iPS cells.

The genes belonging to the Myc family are proto-oncogenes implicated in cancer. It was demonstrated that c-Myc is a factor implicated in the generation of mouse iPS cells and that it was also a factor implicated in the generation of human iPS cells. Introduction of the “Myc” family of genes into target cells for the generation of iPS cells is troubling for the eventuality of iPS cells as clinical therapies, as 25% of mice transplanted with c-Myc-induced iPS cells developed lethal teratomas. N-Myc and 1-Myc have been identified to replace c-myc with similar efficiency.

The term “reprogramming” as used in accordance with the present invention relates to the process of changing the geno- and phenotypical profile of a cell that results in a cell that is geno- and/or phenotypically similar to a stem cell. Said changes comprise, for example, changes in the methylation pattern, shifts in the expression profile or conformational changes of the aggregated nuclear DNA as described herein above.

In particular embodiments, the pluripotency coding sequence represents one or more of a gene selected from one or more of from Oct3 or 4 or a factor belonging to the Myc, Klf and Sox families of factors. In yet other embodiments, the pluripotency coding sequence represents one or more of a gene selected from one or more of more of Oct3, Oct4, 1-Myc, n-Myc, c-Myc, Klf1, Klf2, Klf4, Klf15, Sox1, Sox2, Sox3, Sox15 and Sox18. In specific embodiments, the pluripotency coding sequence represents one or more of a gene selected from one or more of Sox2 or Oct4. In yet another specific embodiment, the coding sequences to be introduced encodes the factors Sox2 and Oct4.

Also, disclosed are construct, vector or cell, and methods that employ them, comprising specific recombination sites or a “recombinase recognition sequence” for corresponding recombinase enzymes. In certain embodiment, the cells of the invention also comprise nucleic acid sequences that encode recognition sequences for recombinases such as Cre/LoxP. Cre recombinase, abbreviated to Cre, is a Type I topoisomerase from P1 bacteriophage that catalyzes site-specific recombination of DNA between loxP sites. In particular embodiments, the pluripotency coding sequence are flanked by loxP sites at the safe-harbor locus.

As used herein a “site-specific endonuclease” is an engineered endonuclease having a recognition region specific to a region in the safe-harbor locus, for attaching and cleaving at that site. In particular embodiments, the site-specific endonuclease comprises a fusion protein comprising a DNA-binding domain and a Fok I cleavage domain or Fok I cleavage domain heterodimer variants, wherein the DNA-binding domain binds to a target site in the safe-harbor locus.

A “fusion” molecule is a molecule in which two or more subunit molecules are linked, preferably covalently. The subunit molecules can be the same chemical type of molecule, or can be different chemical types of molecules. Examples of the first type of fusion molecule include, but are not limited to, fusion proteins (for example, a fusion between a ZFP or TALE DNA-binding domain and a cleavage domain) and fusion nucleic acids (for example, a nucleic acid encoding the fusion protein described supra). Examples of the second type of fusion molecule include, but are not limited to, a fusion between a triplex-forming nucleic acid and a polypeptide, and a fusion between a minor groove binder and a nucleic acid.

Expression of a fusion protein in a cell can result from delivery of the fusion protein to the cell or by delivery of a polynucleotide encoding the fusion protein to a cell, wherein the polynucleotide is transcribed, and the transcript is translated, to generate the fusion protein. Trans-splicing, polypeptide cleavage and polypeptide ligation can also be involved in expression of a protein in a cell. Methods for polynucleotide and polypeptide delivery to cells are presented elsewhere in this disclosure.

A “FokI cleavage domain variant” is a polypeptide sequence which can, in conjunction with a second polypeptide (either identical or different) form a complex having cleavage activity (preferably double-strand cleavage activity). The terms “first and second FokI cleavage domain variant;” “+ and − FokI cleavage domain variant” and “right and left FokI cleavage domain variant” are used interchangeably to refer to pairs of FokI cleavage domain variant that dimerize. In particular embodiments, the FokI cleavage domain variant may be referred to as a half-domain FokI cleavage variant. FokI restriction endonucleases have been described in U.S. Pat. Nos. 5,356,802, 5,436,150, 5,487,994, 5,792,640, 5,916,794, and 6,265,196.

An “engineered FokI cleavage domain variant” is a FokI cleavage domain that has been modified so as to form obligate heterodimers with another FokI cleavage domain variant (e.g., another engineered FokI cleavage domain variant). Engineered FokI cleavage domain variant (also be referred to as dimerization domain mutants) minimize or prevent homodimerization. See International Application No. PCT/US11/45558, filed Jul. 27, 2011, which is incorporated herein by reference in its entirety. Examples of the engineered FokI cleavage domain variant, may include, for example the polypeptide designated D483R:Q486E:I499L, or the polypeptide designated R487D:E490K:I538K.

In particular embodiments, the DNA-binding domain may be zinc finger protein (ZFP) domain or transcription activator-like effector (TALE) domain.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP. Zinc finger binding domains can be “engineered” to bind to a predetermined nucleotide sequence. Non-limiting examples of methods for engineering zinc finger proteins are design and selection. A designed zinc finger protein is a protein not occurring in nature whose design/composition results principally from rational criteria. Rational criteria for design include application of substitution rules and computerized algorithms for processing information in a database storing information of existing ZFP designs and binding data.

Transcription activator-like effector (TALE) protein (or binding domain) is a protein, or a domain within a larger protein, that binds DNA in a sequence-specific manner.

A “selected” or “designer” zinc finger protein or transcription activator-like effector (TALE) protein is a protein not found in nature and is manufactured, e.g., whose production results primarily from an empirical process such as phage display, interaction trap or hybrid selection.

In other embodiments, the site-specific endonuclease may be a zinc finger nuclease (ZFN) or a TALE nuclease (TALEN). Zinc finger nucleases or transcription activator-like effector (TALE) domains can be “engineered” to bind to a predetermined nucleotide sequence.

Examples of designed zinc finger nucleases can be found in US Published Patent Application No. 2010/0055793. Examples of TALE proteins and domains can be found in Miller et al., A TALE nuclease architecture for efficient genome editing, Nat Biotechnol. 2011 February; 29(2):143-8. Epub 2010 Dec. 22; and Hockemeyer et al., Genetic engineering of human pluripotent cells using TALE nucleases, Nat Biotechnol. 2011 Jul. 7. doi: 10.1038/nbt. 1927. See also, Tesson L, Usal C, Ménoret S, Leung E, Niles B J, et al. (2011) Knockout rats generated by embryo microinjection of TALENs. Nature Biotechnology 29:695-696; Sander J D, Cade L, Khayter C, Reyon D, et al. (2011) Targeted gene disruption in somatic zebrafish cells using engineered TALENs. Nature Biotechnology 29:697-698; and Huang P, Xiao A, Zhou M, Zhu Z, et al (2011) Heritable gene targeting in zebrafish using customized TALENs. Nature Biotechnology 29:699-700.

In an exemplary embodiment, the fusion protein includes 3- or 4-zinc finger proteins (ZFP's) or Transcription activator-like effector (TALE) proteins that target CCR5 of human cells, and the obligate heterodimer comprises a first monomer containing the polypeptide designated D483R:Q486E:I499L, and represented by the protein sequence:

(SEQ ID NO. 64)
QLVKSELEEKKSELRHKLKYVPHEYIELIEIARNSTQDRILEMKVMEFFM
KVYGYRGKHLGGSRKPDGAIYTVGSPIDYGVIVDTKAYSGGYNLPIGQAR
EMERYVEENQTRNKHLNPNEWWKVYPSSVTEFKFLFVSGHFKGNYKAQLT
RLNHITNCNGAVLSVEELLIGGEMIKAGTLTLEEVRRKFNNGEINF,

and a second monomer containing the polypeptide designated R487D:E490K:I538K, and represented by the protein sequence:

(SEQ ID NO. 65)
QLVKSELEEKKSELRHKLKYVPHEYIELIEIARNSTQDRILEMKVMEFFM
KVYGYRGKHLGGSRKPDGAIYTVGSPIDYGVIVDTKAYSGGYNLPIGQAD
EMQDYVKENQTRNKHINPNEWWKVYPSSVTEFKFLFVSGHFKGNYKAQLT
RLNHKTNCNGAVLSVEELLIGGEMIKAGTLTLEEVRRKFNNGEINF.

In particular embodiments, the zinc finger proteins (ZFPs) may include ZF1, ZF2, ZF3, ZF4, ZF5 or ZF6, described in Table 1.

TABLE 6
ZF designs for the chosen targets within various mammalian genes

| | | | DNA coding sequence/contact |
|---|---|---|---|
| | ZFN target | Triplet | residues (−1 to +6 positions) |
| | site | subsites | of the α-helix for the ZF |
| Gene | 5′-3′ | 5′-3′ | designs |
| hCCR5 | GCT GCC GCC c | ZF1 GCC c | GAA CGC GGA ACG CTG GCC CGC |
| | (SEQ ID NO: 52) | | (SEQ ID NO: 66) |
| | | | E R G T L A R |
| | | | (SEQ ID NO: 67) |
| | | ZF2 GCC g | GAC CGC TCG GAC TTG ACG CGC |
| | | | (SEQ ID NO: 68) |
| | | | D R S D L T R |
| | | | (SEQ ID NO: 69) |
| | | ZF3 GCT g | CAA TCC TCT GAC TTG ACG CGC |
| | | | (SEQ ID NO: 70) |
| | | | Q S S D L T R |
| | | | (SEQ ID NO: 71) |
| | GAA GGG GAC a | ZF4 GAC a | GAC AGA TCC AAC CTT ACC CGC |
| | (SEQ ID NO: 59) | | (SEQ ID NO: 72) |
| | | | D R S N L T R |
| | | | (SEQ ID NO: 73) |
| | | ZF5 GGG g | CGC AGC GAT CAT CTC ACC AAA |
| | | | (SEQ ID NO: 73) |
| | | | R S D H L T K |
| | | | (SEQ ID NO: 74) |
| | | ZF6 GAA g | CAA TCC TCT AAT CTC GCT CGC |
| | | | (SEQ ID NO: 75) |
| | | | Q S S N L A R |
| | | | (SEQ ID NO: 76) |

A “safe harbor locus” is site within the genome for introducing and/or ectopically expressing other genes as transgenes. That is, functions of safe harbor locus gene or sequence may be expendable, so that the gene or sequence can be cleaved and one of more transgenes of interest can be inserted at the cleavage site. In one embodiment, the safe harbor locus gene can serve to introduce, integrate and/or express one or more other genes or sequences of interest, e.g., pluripotency coding sequences, stem cell factor genes or therapeutic genes.

In some embodiments, the safe-harbor locus may be CCR5 or AAVS1.

CCR5 is described in more detail below. The nonpathogenic human adeno-associated virus (AAV) has developed a mechanism to integrate its genome into human chromosome 19 at 19q13.4 (termed AAVS1), thereby establishing latency. Examples and descriptions of AAVS1 can be found at U.S. Pat. No. 5,580,703 and Dutheil et al. (2000) Proc. Natl. Acad. Sci. USA 97:4862-66, which are hereby incorporated in their entirety.

In particular embodiments, the safe-harbor locus may be CCR5 or AAVS1, the site-specific endonucleases may bind to a target site in the CCR5 or AAVS1 gene, and the CCR5 or AAVS1 gene is cleaved.

In particular embodiments, the safe-harbor locus contains a gene of interest in CCR5, the zinc finger binding domain binds to a target site in the CCR5 gene, and the CCR5 gene is cleaved. In yet other aspects, the zinc finger binding domain comprises, as a recognition region, one of the six sequences shown for hCCR5 in Table 6. In yet another aspect, the recognition region of each of the three zinc fingers is ZF1, ZF2 or ZF3, or ZF4, ZF5 or ZF6.

In particular embodiments, a method is provided, comprising: providing a target somatic cell or primary cell comprising a safe-harbor locus, contacting the cell with a donor construct comprising a reprogramming pluripotency coding sequences and site-specific endonucleases having specificity for a target sequence of interest in the a safe-harbor locus, and culturing the cell to induce reprogramming of the target cell to a stem cell; and culturing the induced stem cell to remove the reprogramming pluripotency coding sequences from the induced stem cell.

In some embodiments of this method, the donor construct is flanked by LoxP site. In yet other aspects, the donor construct is flanked by safe-harbor locus sequence on both the sides for site-specific endonuclease-evoked homology directed repair. In additional embodiments, the safe harbor locus of interest is cleaved and the pluripotency coding sequence is introduced into the genome, thereby giving rise upon transcription to a factor that contributes to the reprogramming of said target cell into an induced pluripotent stem cell.

In embodiments for carrying out the above methods, the method may include adding small molecule inhibitor, valproic acid (VPA), or adding CRE recombinase.

In particular embodiments, the resultant stem cell may be a heterozygous CCR5 single allele mutant resulting homologous recombination (HR). In yet some aspects, the heterozygous hiPSCs has one CCR5 wild type allele and the other CCR5 allele disrupted by a loxP site. In yet different embodiments, the resultant stem cell may be a CCR5 bi-allele mutant resulting from non-homologous end joining (NHEJ). In yet different embodiments, the resultant stem cell may be a CCR5 homozygous mutant, where both alleles are disrupted by loxP sites, In specific aspects, the CRE treated biallele CCR5-disrupted hiPSCs includes one CCR5 allele disrupted NHEJ mutations as prior to CRE treatment and the other CCR5 allele disrupted with a loxP site.

In some embodiments, the disclosed methods may include generation of hiPSCs from human fibroblasts by targeted insertion of Oct4/Sox2 transcription factors at the CCR5 locus using designed ZFNs and VPA. In yet other embodiments, the safe-harbor locus is CCR5 and the site-specific endonucleases comprise a zinc finger binding domain or a TALE binding domain that binds to a target site in the CCR5 gene, and the CCR5 gene is cleaved, and wherein targeted insertion of Oct4/Sox2 transcription factors at the CCR5 locus.

In yet other embodiments, the methods disclosed herein are used to prepare an induced pluripotent stem cell. In yet other aspects, the methods may be used to prepare an enriched population of induced pluripotent stern (iPS) cells. In yet additional embodiments, the methods may be sued to prepare a differentiated cell derived by inducing differentiation of the pluripotent stem cell produced by the disclosed methods.

In other embodiments, a method is provided for generating a transgenic non-human animal comprising the steps of: (a) introducing the induced pluripotent stem cell generated by the above described methods, into a non-human preimplantation embryo; (b) transferring the embryo of step (a) into the uterus of a female non-human animal; and (c) allowing the embryo to develop and to be born. A transgenic non-human animal may be generated by this or other methods.

In yet further embodiments is a composition comprising an induced pluripotent stem cell generated by the above-described methods, for use in gene therapy, regenerative medicine, cell therapy or drug screening.

Also disclosed herein is an isolated or engineered construct, vector or cell comprising a sequence selected from one or more of SEQ ID Nos. 1 to 63. In particular aspects, the isolated or engineered construct, vector or cell comprises a sequence selected from one or more of SEQ ID Nos. 1 to 8 or SEQ ID Nos. 9 to 16 e.g., representing a donor insertion site of heterozygous CCR5-mutant hiPSCs or biallele mutant hiPSCs. In other aspects, the isolated or engineered construct, vector or cell comprises a sequence selected from one or more of SEQ ID Nos. 17 to 20 e.g., representing a nucleotide sequence of mutant CCR5 locus of bi-allele mutant hiPSCs. In yet further aspects, the isolated or engineered construct, vector or cell comprises a sequence selected from one or more of SEQ ID Nos. 21 to 28 e.g., representing a nucleotide sequence of mutant CCR5 mutant allele in heterozygous hiPSCs. In some aspects, the isolated or engineered construct, vector or cell comprises a sequence selected from one or more of SEQ ID Nos. 29 to 36 e.g., representing a nucleotide sequence of CCR5 locus in biallelic mutant hiPSCs. In other aspects, the isolated or engineered construct, vector or cell comprises a sequence selected from one or more of SEQ ID Nos. SEQ ID NOS: 37, 38, 39, 40, and 41, e.g., representing the endogenous CCR5 locus of HEK293 cell lines expressing the WT_CFTR and mutant CFTRΔ508F protein, respectively. In yet other aspects, the isolated or engineered construct, vector or cell comprises a sequence selected from one or more of SEQ ID Nos. 42 to 46, e.g., representing sequences of the tdTomato/CFTR donor insertion site in heterozygous CCR5-mutant hiPSCs. In yet additional aspects, the isolated or engineered construct, vector or cell comprises a sequence selected from one or more of SEQ ID Nos. SEQ ID Nos. 47 to 51, e.g., representing sequences of the tdTomato/CFTR donor insertion site in heterozygous CCR5-mutant hiPSCs. In other aspects, the isolated or engineered construct, vector or cell comprises a sequence selected from one or more of SEQ ID Nos. 52 to 63, e.g., representing sequences of CCR5.

In yet other embodiments, a donor DNA construct or molecule is provided with one or more stem cell factor gene or sequences and recombinase recognition sequence, e.g., a LoxP sequence, wherein the one or more stem cell factor gene or sequences and a recombinase recognition sequence, LoxP sequence, are flanked by a safe-harbor locus sequence on both the sides. In yet other embodiments, a DNA construct or molecule is provided having (a) a first DNA sequence comprising: a coding sequence giving rise upon transcription to a factor that contributes to the reprogramming of a somatic cell or primary cell into an induced pluripotent stem cell; a promoter mediating the transcription of said coding sequence; (b) a recombinase recognition sequence, e.g., LoxP sequence, and (c) second DNA sequence comprising a safe-harbor locus sequence mediates site-specific integration of (a) into another DNA molecule or a target genome.

In particular aspects of the donor DNA construct, the safe-harbor locus may be CCR5 or AAVS1. In yet other aspects, the one or more stem cell factor gene or sequences are flanked by the recombinase recognition sequence, e.g., LoxP sequence, on both the sides. In yet a further aspect, the stem cell factor gene or sequence or the coding sequence giving rise upon transcription represents one or more of a gene selected from one or more of Sox2 or Oct4. In further embodiments, the one or more of a genes are selected from one or more of Sox2 or Oct4 that are flanked by recombinase recognition sites, e.g., LoxP sites, at a CCR5 safe-harbor locus.

Also, disclosed are constructs, vectors or cells, and methods that employ them, comprising a drug selection gene or a fluorescent marker gene or both. As used herein a “selection or marker gene” may include fluorescent marker genes, such as tdTomato or eGFP, or a drug selection gene, such as Puromycin or Hygromycin. In certain embodiments, the fluorescent marker gene is tdTomato or eGFP sequence.

In certain embodiments, the construct may further include a therapeutic gene and a selection or marker gene, e.g., tdTomato sequence.

In other embodiment, a donor DNA construct or molecule is provided having one or more of a therapeutic gene or selection or marker gene, e.g., tdTomato sequence, wherein the one or more of a therapeutic gene or sequence and a selection or marker gene, e.g., tdTomato sequence, are flanked by a safe-harbor locus sequence on both the sides. In particular aspects, the safe-harbor locus may be CCR5 or AAVS1. In some embodiments, the therapeutic gene or sequence is wild-type CFTR. In other embodiments, the therapeutic gene is beta globin gene or sequence.

Also provided are vectors containing the above-described donor DNA constructs.

In some embodiments, a method is provided for generating an induced pluripotent stem cell comprising the steps of: (i) introducing a site-specific endonucleases, such as ZFNs or TALENs, along with the DNA construct(s) or the vector(s) described above, into a somatic cell; (ii) allowing the DNA construct(s) or the vector(s) of step (i) to integrate into the genomic DNA of the somatic cell; and (iii) excising the stem cell factor gene or sequences from the DNA molecule, wherein step (iii) is performed after reprogramming of said somatic cell has taken place.

Also disclosed herein is an induced pluripotent stem cell obtainable by the method described above. Moreover, a cell line or cell culture collection containing the induced pluripotent stem cell derived from the above methods is also provided. In yet other aspects, a differentiated cell is derived by inducing differentiation of the pluripotent stem cell produced by the above-methods or from the above constructs or vectors.

In yet other embodiments, a kit is provided containing (in separate packaging or the same) the DNA construct(s) or components thereof, the genes and sequences, the vector(s), or the induced pluripotent cell or cells, as described above.

A method to generate a transgenic non-human animal comprising (i) introducing the induced pluripotent stem cells prepared from the constructs vectors described above, into a non-human blastocyst; (ii) transferring the blastocyst into the uterus of a female non-human animal; and (iii) allowing the blastocyst to develop into an embryo. Disclosed herein is a transgenic non-human animal obtainable by the above-described methods.

Also provided is composition containing the cells obtained by the above-described methods, for gene therapy, regenerative medicine, cell therapy or drug screening.

In further embodiments, disclosed herein are methods of treating a variety of conditions using the constructs, vectors, cells, compositions prepared from the above methods.

In particular embodiments, a method of treating a subject is provided, comprising: a) generating an induced pluripotent stem (iPS) cell from a somatic cell or primary cell of the subject by the method above methods; b) inducing differentiation of the iPS cell of step (a); and c) introducing the cell of (b) into the subject, thereby treating the condition.

In some embodiments, particular conditions are genetic diseases, such as monogenic or autosomal recessive allele disorders.

Exemplary genetic diseases include, but are not limited to, achondroplasia, achromatopsia, acid maltase deficiency, adenosine deaminase deficiency (OMIM No. 102700), adrenoleukodystrophy, aicardi syndrome, alpha-1 antitrypsin deficiency, alpha-thalassemia, androgen insensitivity syndrome, apert syndrome, arrhythmogenic right ventricular, dysplasia, ataxia telangictasia, barth syndrome, beta-thalassemia, blue rubber bleb nevus syndrome, canavan disease, chronic granulomatous diseases (CGD), cri du chat syndrome, cystic fibrosis, dercum's disease, ectodermal dysplasia, fanconi anemia, fibrodysplasia ossificans progressive, fragile X syndrome, galactosemis, Gaucher's disease, generalized gangliosidoses (e.g., GM1), hemochromatosis, the hemoglobin C mutation in the 6^{th} codon of beta-globin (HbC), hemophilia, Huntington's disease, Hurler Syndrome, hypophosphatasia, Klinefleter syndrome, Krabbes Disease, Langer-Giedion Syndrome, leukocyte adhesion deficiency (LAD, OMIM No. 116920), leukodystrophy, long QT syndrome, Marfan syndrome, Moebius syndrome, mucopolysaccharidosis (MPS), nail patella syndrome, nephrogenic diabetes insipdius, neurofibromatosis, Neimann-Pick disease, osteogenesis imperfecta, porphyria, Prader-Willi syndrome, progeria, Proteus syndrome, retinoblastoma, Rett syndrome, Rubinstein-Taybi syndrome, Sanfilippo syndrome, severe combined immunodeficiency (SCID), Shwachman syndrome, sickle cell disease (sickle cell anemia), Smith-Magenis syndrome, Stickler syndrome, Tay-Sachs disease, Thrombocytopenia Absent Radius (TAR) syndrome, Treacher Collins syndrome, trisomy, tuberous sclerosis, Turner's syndrome, urea cycle disorder, von Hippel-Landau disease, Waardenburg syndrome, Williams syndrome, Wilson's disease, Wiskott-Aldrich syndrome, X-linked lymphoproliferative syndrome (XLP, OMIM No. 308240).

Some autosomal recessive allele disorders may include sickle-cell disease, Tay-Sachs disease, Niemann-Pick disease, spinal muscular atrophy, or Roberts syndrome.

In particular embodiments, methods are disclosed for site-specific integration of therapeutic genes for treating an autosomal recessive allele disorders, comprising introducing into the target cell one or more of therapeutic gene sequences at a safe-harbor locus within the cell target genome using a site-specific endonuclease. In particular embodiments, the therapeutic gene is wild-type CFTR and the autosomal recessive allele disorder is cystic fibrosis. In yet other embodiments, the therapeutic gene is beta globin gene and the autosomal recessive allele disorder is sickle-cell disease (SCD). In some embodiments, the site-specific endonucleases may be a zinc finger nuclease (ZFN) or a TALE nuclease (TALEN), and the safe-harbor locus may be CCR5 or AAVS1. In particular embodiments, the safe-harbor locus is CCR5 or AAVS1, the site-specific endonucleases bind to a target site in the CCR5 or AAVS1 gene, and the CCR5 or AAVS1 gene is cleaved. In these embodiments, the target cell may be human induced pluripotent stem cells (hiPSC), human adult stem cells, human stem progenitor cells (HSPC) or cord blood cells. In particular embodiments, the hiPSC contains a heterozygous CCR5 single allele mutant. In other embodiments, the heterozygous hiPSCs has one CCR5 wild type allele and the other CCR5 allele mutant allele disrupted by a loxP site.

In particular embodiments, methods are provided including: providing a target cell comprising the safe-harbor locus, contacting a cell with a donor construct comprising a therapeutic gene and constructs for site-specific endonucleases having specificity for a sequence of interest in the safe-harbor locus, and culturing the cell to integrate the therapeutic gene at the safe-harbor locus. In some aspects, the therapeutic gene is integrated at safe-harbor locus by non-homologous end joining (NHEJ). In some embodiments, the donor construct is flanked by safe-harbor locus sequence on both the sides for site-specific endonuclease-evoked homology directed repair. In further embodiments, the gene of interest is cleaved and the therapeutic gene is introduced into the genome. In yet additional embodiments, the safe-harbor locus is CCR5 and the site-specific endonuclease comprises a zinc finger binding domain that binds to a target site in the CCR5 gene, and the CCR5 gene is cleaved, and wherein the wild-type CFTR is inserted at the CCR5 locus.

In particular embodiments, a method of treating a subject for cystic fibrosis is provided, comprising: a) generating an induced pluripotent stem (iPS) cell comprising the therapeutic gene wild-type CFTR from a somatic cell or primary cell of the subject by the method above methods; b) inducing differentiation of the iPS cell of step (a); and c) introducing the cell of (b) into the subject, thereby treating the cystic fibrosis.

In particular embodiments, a method of treating a subject for sickle-cell disease (SCD) is provided, comprising: a) generating an induced pluripotent stem (iPS) cell comprising beta globin gene from a somatic cell or primary cell of the subject by the method above methods; b) inducing differentiation of the iPS cell of step (a); and c) introducing the cell of (b) into the subject, thereby treating the sickle-cell disease (SCD).

Other conditions or diseases that may be treated by the present compositions by targeted DNA cleavage and/or homologous recombination include acquired immunodeficiencies, lysosomal storage diseases (e.g., Gaucher's disease, GM 1, Fabry disease and Tay-Sachs disease), mucopolysaccahidosis (e.g. Hunter's disease, Hurler's disease), hemoglobinopathies (e.g., sickle cell diseases, HbC, α-thalassemia, β-thalassemia) and hemophilias. Such methods also allow for treatment of infections (viral or bacterial) in a host (e.g., by blocking expression of viral or bacterial receptors, thereby preventing infection and/or spread in a host organism); to treat genetic diseases.

Targeted cleavage of infecting or integrated viral genomes can be used to treat viral infections in a host. Additionally, targeted cleavage of genes encoding receptors for viruses can be used to block expression of such receptors, thereby preventing viral infection and/or viral spread in a host organism. Targeted mutagenesis of genes encoding viral receptors (e.g., the CCR5 and CXCR4 receptors for HIV) can be used to render the receptors unable to bind to virus, thereby preventing new infection and blocking the spread of existing infections.

Non-limiting examples of viruses or viral receptors that may be targeted include herpes simplex virus (HSV), such as HSV-1 and HSV-2, varicella zoster virus (VZV), Epstein-Barr virus (EBV) and cytomegalovirus (CMV), HHV6 and HHV7. The hepatitis family of viruses includes hepatitis A virus (HAV), hepatitis B virus (HBV), hepatitis C virus (HCV), the delta hepatitis virus (HDV), hepatitis E virus (HEV) and hepatitis G virus (HGV). Other viruses or their receptors may be targeted, including, but not limited to, Picornaviridae (e.g., polioviruses, etc.); Caliciviridae; Togaviridae (e.g., rubella virus, dengue virus, etc.); Flaviviridae; Coronaviridae; Reoviridae; Bimaviridae; Rhabodoviridae (e.g., rabies virus, etc.); Filoviridae; Paramyxoviridae (e.g., mumps virus, measles virus, respiratory syncytial virus, etc.); Orthomyxoviridae (e.g., influenza virus types A, B and C, etc.); Bunyaviridae; Arenaviridae; Retroviradae; lentiviruses (e.g., HTLV-I; HTLV-II; HIV-1 (also known as HTLV-III, LAV, ARV, hTLR, etc.) HIV-II); simian immunodeficiency virus (SIV), human papillomavirus (HPV), influenza virus and the tick-borne encephalitis viruses. See, e.g. Virology, 3rd Edition (W. K. Joklik ed. 1988); Fundamental Virology, 2nd Edition (B. N. Fields and D. M. Knipe, eds. 1991), for a description of these and other viruses. Receptors for HIV, for example, include CCR5 and CXCR-4.

Among the genes which can be cleaved is CCR5 co-receptor (hCCR5) through which HIV gains entry into cells early in the infection. Thus, in one aspect, described herein are compositions and methods useful for disrupting the CCR5 gene in cells comprising an engineered fusion protein including zinc finger binding domain or transcription activator-like effector (TALE) domain to bind to a CCR5 target sequence and an engineered FokI cleavage domain variant, wherein said fusion protein binds to the CCR5 gene and cleaves the CCR5 gene. The mutation can be associated with any function of CCR5, e.g. the ability of an HIV virus to enter a host cell via the CCR5 co-receptor. CCR5 genes can be disrupted for a variety of purposes. For example, after cleavage of CCR5, the gene can be repaired by non-homologous end-joining in the cell to give rise to a CCR5 gene mutation that inactivates the CCR5 receptor to produce HIV resistant cells. Alternatively, CCR5 receptor can be disrupted by replacing a wild type sequence with a CCR5Δ32 mutation. In one embodiment, a CCR5 chromosomal gene locus can serve as a “safe harbor” for the introduction of transgenes. That is, functions of CCR5 may be expendable, so that the gene can be cleaved and one of more therapeutic transgenes of interest can be inserted at the cleavage site for functional complementation in cells. In one embodiment, the CCR5 gene is a human gene, where one or more genes of interest can be introduced and expressed ectopically. These genes can be marker genes (e.g. neomycin or green fluorescent protein (GFP)) or genes applicable for human therapeutics.

In particular embodiments a differentiated cell is derived by inducing differentiation of the pluripotent stem cell having a CCR5 bi-allele mutant or CCR5-disrupted homozygous mutant allele produced by the above methods and constructs or vectors. These cells may be a homogeneous population of HIV-resistant cells. In some aspects, the stem cell is hiPSC with disrupted bi-allele CCR5 or CCR5-disrupted homozygous mutant allele that are resistant to HIV infection. In yet other embodiments, the bi-allele CCR5-disrupted hiPSCs or CCR5-disrupted homozygous mutant allele is permanently resistant to HIV. In particular embodiments, hiPSC is differentiated into CD34+ haematopoietic cell. In further embodiments, isolated single cell CCR5-modified hiPSC colonies are differentiated into HIV resistant CD34+ hematopoietic stem cells (HSCs) and expanded in vitro for transplantation. In yet other aspects, disclosed are a uniform biallele CCR5-disrupted cell population containing a specific mutation from isolated single cell CCR5-modified hiPSC colonies.

In particular embodiments, a method of treating reducing and/or curing HIV-1 infection in a subject is provided, comprising: a) generating an induced pluripotent stem (iPS) cell comprising having a CCR5 bi-allele mutant or CCR5 homozygous mutant allele from a somatic cell or primary cell of the subject by the method above methods; b) inducing differentiation of the iPS cell of step (a) to CD34+ haematopoietic cell; and c) introducing the cell of (b) into the subject, thereby treating, reducing and/or curing HIV-1 infection in the subject.

Conventional viral and non-viral based gene transfer methods can be used to introduce sequences, genes, constructs, vectors and cells (including those encoding ZFNs or TALENs) in cells (e.g., mammalian cells) and target tissues. Such methods can also be used to administer such nucleic acids to cells in vitro. In certain embodiments, sequences, genes, constructs, vectors and cells (including those encoding ZFNs or TALENs) are administered for in vivo or ex vivo gene therapy uses. Non-viral vector delivery systems include DNA plasmids, naked nucleic acid, and nucleic acid complexed with a delivery vehicle such as a liposome or poloxamer. Viral vector delivery systems include DNA and RNA viruses, which have either episomal or integrated genomes after delivery to the cell. For a review of gene therapy procedures, see Anderson, Science 256:808-813 (1992); Nabel & Feigner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bohm (eds.) (1995); and Yu et al., Gene Therapy 1:13-26 (1994).

Methods of non-viral delivery of sequences, genes, constructs, vectors and cells (including those encoding ZFNs or TALENs) include electroporation, lipofection, microinjection, biolistics, virosomes, liposomes, immunoliposomes, polycation or lipid:nucleic acid conjugates, naked DNA, artificial virions, and agent-enhanced uptake of DNA. Sonoporation using, e.g., the Sonitron 2000 system (Rich-Mar) can also be used for delivery of nucleic acids. Additional exemplary nucleic acid delivery systems include those provided by Amaxa Biosystems (Cologne, Germany), Maxcyte, Inc. (Rockville, Md.) and BTX Molecular Delivery Systems (Holliston, Mass.). Lipofection and lipofection reagents are sold commercially (e.g., Transfectam™ and Lipofectin™). Cationic and neutral lipids that are suitable for efficient receptor-recognition lipofection of polynucleotides include those of Feigner, WO 91/17424, WO 91/16024. Delivery can be to cells (ex vivo administration) or target tissues (in vivo administration).

The preparation of lipid nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); and Ahmad et al., Cancer Res. 52:4817-4820 (1992).

The use of RNA or DNA viral based systems for the delivery of sequences, genes, constructs, vectors and cells (including those encoding ZFNs or TALENs) comprising constructs as described herein take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of sequences, genes, constructs, vectors and cells (including those encoding ZFNs or TALENs) include, but are not limited to, retroviral, lentivirus, adenoviral, adeno-associated, vaccinia and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors, including integration deficient lentiviral vectors (IDLV) are retroviral vectors that are able to transduce or infect non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (see, e.g., Buchscher et al., J. Virol. 66:2731-2739 (1992); Johann et al., J. Virol. 66:1635-1640 (1992); Sommerfelt et al., Virol. 176:58-59 (1990); Wilson et al., J. Virol. 63:2374-2378 (1989); Miller et al., J. Virol. 65:2220-2224 (1991); PCT/US94/05700).

In applications in which transient expression of a pair of ZFN or TALEN fusion proteins is preferred, adenoviral based systems can be used. Adenoviral based vectors are capable of very high transduction efficiency in many cell types and do not require cell division. With such vectors, high titer and high levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (Dunbar et al., Blood 85:3048-305 (1995); Kohn et al., Nat. Med. 1:1017-102 (1995); Malech et al., PNAS 94:22 12133-12138 (1997)). PA317/pLASN was the first therapeutic vector used in a gene therapy trial. (Blaese et al., Science 270:475-480 (1995)). Transduction efficiencies of 50% or greater have been observed for MFG-S packaged vectors. (Ellem et al., Immunol Immunother. 44(1):10-20 (1997); Dranoffet al., Hum. Gene Ther. 1:111-2 (1997).

Recombinant adeno-associated virus vectors (rAAV) are a promising alternative gene delivery systems based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system. (Wagner et al., Lancet 351:9117 1702-3 (1998), Kearns et al., Gene Ther. 9:748-55 (1996)).

Replication-deficient recombinant adenoviral vectors (Ad) can be produced at high titer and readily infect a number of different cell types. Most adenovirus vectors are engineered such that a transgene replaces the Ad E1a, E1b, and/or E3 genes; subsequently the replication defective vector is propagated in human 293 cells that supply deleted gene function in trans. Ad vectors can transduce multiple types of tissues in vivo, including nondividing, differentiated cells such as those found in liver, kidney and muscle. Conventional Ad vectors have a large carrying capacity. An example of the use of an Ad vector in a clinical trial involved polynucleotide therapy for antitumor immunization with intramuscular injection (Sterman et al., Hum. Gene Ther. 7:1083-9 (1998)). Additional examples of the use of adenovirus vectors for gene transfer in clinical trials include Rosenecker et al., Infection 24:1 5-10 (1996); Sterman et al., Hum. Gene Ther. 9:7 1083-1089 (1998); Welsh et al., Hum. Gene Ther. 2:205-18 (1995); Alvarez et al., Hum. Gene Ther. 5:597-613 (1997); Topfet al., Gene Ther. 5:507-513 (1998); Sterman et al., Hum. Gene Ther. 7:1083-1089 (1998).

In certain embodiments, the vector is an adenovirus vector. Thus, described herein are adenovirus (Ad) vectors for introducing heterologous sequences (e.g., ZFNs or TALENs) into cells.

Non-limiting examples of Ad vectors that can be used in the present application include recombinant (such as E1-deleted), conditionally replication competent (such as oncolytic) and/or replication competent Ad vectors derived from human or non-human serotypes (e.g., Ad5, Ad11, Ad35, or porcine adenovirus-3); and/or chimeric Ad vectors (such as Ad5/35) or tropism-altered Ad vectors with engineered fiber (e.g., knob or shaft) proteins (such as peptide insertions within the HI loop of the knob protein). Also useful are “gutless” Ad vectors, e.g., an Ad vector in which all adenovirus genes have been removed, to reduce immunogenicity and to increase the size of the DNA payload. This allows, for example, simultaneous delivery of sequences encoding ZFNs or TALENs and a donor sequence. Such gutless vectors are especially useful when the donor sequences include large transgenes to be integrated via targeted integration.

Replication-deficient recombinant adenoviral vectors (Ad) can be produced at high titer, and they readily infect a number of different cell types. Most adenovirus vectors are engineered such that a transgene replaces the Ad E1a, E1b, and/or E3 genes; subsequently the replication defective vector is propagated in cells that provide one or more of the deleted gene functions in trans. For example, human 293 cells supply E1 function. Ad vectors can transduce multiple types of tissues in vivo, including non-dividing, differentiated cells such as those found in liver, kidney and muscle. Conventional Ad vectors have a large carrying capacity. An example of the use of an Ad vector in a clinical trial involved polynucleotide therapy for antitumor immunization with intramuscular injection (Sterman et al., Hum. Gene Ther. 7:1083-1089 (1998)).

Additional examples of the use of adenovirus vectors for gene transfer in clinical trials include Rosenecker et al., Infection 24:1 5-10 (1996); Welsh et al., Hum. Gene Ther. 2:205-18 (1995); Alvarez et al., Hum. Gene Ther. 5:597-613 (1997); Topf et al., Gene Ther. 5:507-513 (1998).

In certain embodiments, the Ad vector is a chimeric adenovirus vector, containing sequences from two or more different adenovirus genomes. For example, the Ad vector can be an Ad5/35 vector. Ad5/35 is created by replacing one or more of the fiber protein genes (knob, shaft, tail, penton) of Ad5 with the corresponding fiber protein gene from a B group adenovirus such as, for example, Ad35. The Ad5/35 vector and characteristics of this vector are described, for example, in Ni et al. (2005) “Evaluation of biodistribution and safety of adenovirus vectors containing group B fibers after intravenous injection into baboons,” Hum Gene Ther 16:664-677; Nilsson et al. (2004) “Functionally distinct subpopulations of cord blood CD34+ cells are transduced by adenoviral vectors with serotype 5 or 35 tropism,” Mol Ther 9:377-388; Nilsson et al. (2004) “Development of an adenoviral vector system with adenovirus serotype 35 tropism; efficient transient gene transfer into primary malignant hematopoietic cells,” J Gene Med 6:631-641; Schroers et al. (2004) “Gene transfer into human T lymphocytes and natural killer cells by Ad5/F35 chimeric adenoviral vectors,” Exp Hematol 32:536-546; Seshidhar et al. (2003) “Development of adenovirus serotype 35 as a gene transfer vector,” Virology 311:384-393; Shayakhmetov et al. (2000) “Efficient gene transfer into human CD34(+) cells by a retargeted adenovirus vector,” J Virol 74:2567-2583; and Soya et al. (2004), “A tumor-targeted and conditionally replicating oncolytic adenovirus vector expressing TRAIL for treatment of liver metastases,” Mol Ther 9:496-509.

Packaging cells are used to form virus particles that are capable of infecting a host cell. Such cells include 293 cells, which package adenovirus, and ψ2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al., Proc. Natl. Acad. Sci. USA 92:9747-9751 (1995), reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a ZFNs or TALENs nucleic acid (gene or cDNA), and re-infused back into the subject organism (e.g., patient). Various cell types suitable for ex vivo transfection are well known to those of skill in the art (see, e.g., Freshney et al., Culture of Animal Cells, A Manual of Basic Technique (3rd ed. 1994)) and the references cited therein for a discussion of how to isolate and culture cells from patients).

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-γ and TNF-α are known (see Inaba et al., J. Exp. Med. 176:1693-1702 (1992)).

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+ (panB cells), GR-1 (granulocytes), and lad (differentiated antigen presenting cells) (see Inaba et al., J. Exp. Med. 176:1693-1702 (1992)).

Vectors (e.g., retroviruses, adenoviruses, liposomes, etc.) containing therapeutic ZFNs or TALENs nucleic acids can also be administered directly to an organism for transduction of cells in vivo. Alternatively, naked DNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Methods for introduction of DNA into hematopoietic stem cells are known and vectors useful for introduction of transgenes into hematopoietic stem cells, e.g., CD34+ cells, include adenovirus Type 35. Vectors suitable for introduction of transgenes into immune cells (e.g., T-cells) include non-integrating lentivirus vectors. See, for example, Ory et al. (1996) Proc. Natl. Acad. Sci. USA 93:11382-11388; Dull et al. (1998) J. Virol. 72:8463-8471; Zuffery et al. (1998) J. Virol. 72:9873-9880; Follenzi et al. (2000) Nature Genetics 25:217-222.

Pharmaceutically acceptable carriers are determined in part by the particular composition being administered, as well as by the particular method used to administer the composition. Accordingly, there is a wide variety of suitable formulations of pharmaceutical compositions available, as described below (see, e.g., Remington's Pharmaceutical Sciences, 17th ed., 1989).

As noted above, the disclosed methods and compositions can be used in any type of cell including, but not limited to, prokaryotic cells, fungal cells, Archaeal cells, plant cells, insect cells, animal cells, vertebrate cells, mammalian cells and human cells. Suitable cell lines for protein expression are known to those of skill in the art and include, but are not limited to COS, CHO (e.g., CHO-S, CHO-K1, CHO-DG44, CHO-DUXB11), VERO, MDCK, WI38, V79, B14AF28-G3, BHK, HaK, NS0, SP2/0-Ag14, HeLa, HEK293 (e.g., HEK293-F, HEK293-H, HEK293-T), perC6, insect cells such as Spodoptera fugiperda (Sf), and fungal cells such as Saccharomyces, Pischia and Schizosaccharomyces. Progeny, variants and derivatives of these cell lines can also be used.

The disclosed compositions can be used to cleave at a region of interest in cellular chromatin (e.g., at a desired or predetermined site in a genome, for example, in a gene, either mutant or wild-type); to replace a genomic sequence (e.g., a region of interest in cellular chromatin) with a homologous non-identical sequence (i.e., targeted recombination); to delete a genomic sequence by cleaving DNA at one or more sites in the genome, which cleavage sites are then joined by non-homologous end joining (NHEJ); to screen for cellular factors that facilitate homologous recombination; and/or to replace a wild-type sequence with a mutant sequence, or to convert one allele to a different allele.

Accordingly, the disclosed engineered compositions can be used for any method in which specifically targeted cleavage is desirable and/or to replace any genomic sequence with a homologous, non-identical sequence. For example, a mutant genomic sequence can be replaced by its wild-type counterpart, thereby providing methods for treatment of e.g., genetic disease, inherited disorders, cancer, and autoimmune disease. In like fashion, one allele of a gene can be replaced by a different allele using the methods of targeted recombination disclosed herein. Indeed, any pathology dependent upon a particular genomic sequence, in any fashion, can be corrected or alleviated using the methods and compositions disclosed herein.

As noted above, the compositions and methods described herein can be used for gene modification, gene correction, and gene disruption. Non-limiting examples of gene modification includes homology directed repair (HDR)-based targeted integration; HDR-based gene correction; HDR-based gene modification; HDR-based gene disruption; NHEJ-based gene disruption and/or combinations of HDR, NHEJ, and/or single strand annealing (SSA). Single-Strand Annealing (SSA) refers to the repair of a double strand break between two repeated sequences that occur in the same orientation by resection of the DSB by 5′-3′ exonucleases to expose the 2 complementary regions. The single-strands encoding the 2 direct repeats then anneal to each other, and the annealed intermediate can be processed such that the single-stranded tails (the portion of the single-stranded DNA that is not annealed to any sequence) are be digested away, the gaps filled in by DNA Polymerase, and the DNA ends rejoined. This results in the deletion of sequences located between the direct repeats.

Compositions comprising the constructs, vectors, cells and cleavage domains (e.g., ZFNs or TALENs) and methods described herein can also be used in the treatment of various genetic diseases and/or infectious diseases.

The compositions and methods can also be applied to stem cell based therapies, including but not limited to: (a) Correction of somatic cell mutations by short patch gene conversion or targeted integration for monogenic gene therapy; (b) Disruption of dominant negative alleles; (c) Disruption of genes required for the entry or productive infection of pathogens into cells; (d) Enhanced tissue engineering, for example, by: (i) Modifying gene activity to promote the differentiation or formation of functional tissues, and/or (ii) Disrupting gene activity to promote the differentiation or formation of functional tissues; (e) Blocking or inducing differentiation, for example, by: (i) Disrupting genes that block differentiation to promote stem cells to differentiate down a specific lineage pathway, (ii) Targeted insertion of a gene or siRNA expression cassette that can stimulate stem cell differentiation, (iii) Targeted insertion of a gene or siRNA expression cassette that can block stem cell differentiation and allow better expansion and maintenance of pluripotency, and/or (iv) Targeted insertion of a reporter gene in frame with an endogenous gene that is a marker of pluripotency or differentiation state that would allow an easy marker to score differentiation state of stem cells and how changes in media, cytokines, growth conditions, expression of genes, expression of siRNA molecules, exposure to antibodies to cell surface markers, or drugs alter this state; (f) Somatic cell nuclear transfer, for example, a patient's own somatic cells can be isolated, the intended target gene modified in the appropriate manner, cell clones generated (and quality controlled to ensure genome safety), and the nuclei from these cells isolated and transferred into unfertilized eggs to generate patient-specific hES cells that could be directly injected or differentiated before engrafting into the patient, thereby reducing or eliminating tissue rejection; and/or (g) Universal stem cells by knocking out MHC receptors—this approach would be used to generate cells of diminished or altogether abolished immunological identity. Cell types for this procedure include but are not limited to, T-cells, B cells, hematopoietic stem cells, and embryonic stem cells. Therefore, these stem cells or their derivatives (differentiated cell types or tissues) could be potentially engrafted into any person regardless of their origin or histocompatibility. (h) Targeted insertion of stem cell factor genes at a safe-harbor locus (CCR5 locus or AAVS1 site located on human chromosome PPP1R12C gene) within the human genome to reprogram cells to form induced pluripotent stem cells. (i) Targeted addition of therapeutic genes at a safe-harbor locus (CCR5 locus or AAVS1 site located on human chromosome PPP1R12C gene) within the human genome to provide functional protein complementation in cells with corresponding defective genes. (j) Targeted disruption of CCR5 by HDR or NHEJ to produce HIV resistant cells. (k) Genetic engineering of human pluripotent stem cells.

The compositions and methods can also be used for somatic cell therapy (e.g., autologus cell therapy and/or universal T-cell by knocking out MHC receptors, see section (g) above), thereby allowing production of stocks of T-cells that have been modified to enhance their biological properties. Such cells can be infused into a variety of patients independent of the donor source of the T-cells and their histocompatibility to the recipient.

In addition to therapeutic applications, the increased specificity provided by the variants described herein when used in ZFNs or TALENs can be used for crop engineering, cell line engineering and the construction of disease models.

The constructs, vectors and cells described can also be used in gene modification protocols requiring simultaneous cleavage at multiple targets either to delete the intervening region or to alter two specific loci at once. Cleavage at two targets would require cellular expression of four ZFNs or TALENs, which would yield ten different active ZFN or TALEN combinations. For such applications, substitution of our variants for the wild-type nuclease domain would eliminate the activity of six of these combinations and reduce chances of off-target cleavage.

As used herein, the DNA and other nucleic acid with sequences, polypeptides, proteins, antibodies, constructs, vectors or cells, embodiment in the invention, are “isolated” and therefore have structurally and chemically distinct features from what is found in nature or is otherwise simply purified. In some embodiment, these products are “isolated” versions and may differ from those existing in nature or those that are just “purified”. In some embodiments, the products comprise a modified version of the nucleic acid and/or any nucleic acid of interest plus something else. Examples include cDNA and non-naturally occurring sequences (even if different by one nucleotide), hybrid and fusion molecules, vectors, recombinant cells, labeled nucleic acids (e.g., probes), nucleic acids in combination with a reagent or buffer or any other “non-naturally” occurring combinations, assays and kits comprising the nucleic acid (e.g., primer) in a relevant form. Exemplary embodiments also include polypeptides or proteins with non-naturally occurring sequences (even if different by a single amino acid), as well as molecules missing or gaining a glycosylation, methylation or other chemical modification, as compared to a native molecule. Also included are relevant hybrid and fusion molecules, vectors and/or recombinant cells expressing the polypeptide or protein, labeled molecules, molecules in combination with a reagent or any other “non-naturally” occurring combinations (e.g., polypeptide plus an excipient, inactive ingredient, buffer, storage solution, etc.), assays and kits comprising the molecule in a relevant form.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The invention is to be understood as not being limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

All publications mentioned herein, including patents, published patent applications, and journal articles are incorporated herein by reference in their entireties including the references cited therein, which are also incorporated herein by reference.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The patent or application file contains at least one drawing executed in color. Copies of this patent or patent application publication with color drawing(s) will be provided by the Office upon request and payment of the necessary fee.

This application claims priority to U.S. provisional application No. 61/372,166, filed Aug. 10, 2010, which is hereby incorporated herein by reference in their entirety.

### EXAMPLES

The following examples detail methods and compositions for generating hiPSCs using designed ZFNs, followed by the ability to regulate and reshape their functionality by targeted addition of therapeutic genes at the endogenous CCR5 locus of the human genome using designed ZFNs for ectopic expression and functional complementation. These have enormous potential not only for basic biomedical research, but also for human therapeutics and regenerative medicine in the future.

In these examples, we have used the well-characterized highly specific 4-finger ZFNs, which were previously shown not to result in have additional integrations. Southern blot analysis was used to exclude additional integrations. ZFN-mediated double strand breaks and error-prone repair by NHEJ elsewhere in the genome needs to be examined in great detail on a genome wide basis, particularly if human therapeutics is being contemplated. This may necessitate very detailed analysis of multiple single cell hiPSC colonies before the correct one could be identified.

In summary, this novel approach to generate hiPSCs using designed ZFNs (or designed TALENs) and the ability to regulate and reshape the functionality of hiPSCs by targeted addition of therapeutic genes at the endogenous CCR5 safe-harbor locus of the human genome for functional complementation, has enormous potential for basic biomedical research in the near future and human therapeutics in the future.

### Example 1

### Materials and Methods

### Construction of ZFN Plasmids and Donor Plasmid Containing the Reprogramming Transcription Factor Genes

The open reading frames (ORFs) of human Oct4, Sox2 were amplified from cDNA clones obtained from Harvard Proteomics. The PCR amplified DNAs were placed between Foot and Mouth disease virus 2A sequences which allows efficient polycistronic expression. A single CMV promoter, which drives the expression of these two transgenes, along with marker genes (either eGFP or puromycin resistance gene) were cloned into pNTKV using AflII and BamHI restriction sites. The transgene cassette was flanked by LoxP sites for cre/lox-mediated excision of the transgenic cassette. This whole transgene cassette was flanked by 750 bp endogeneous CCR5 locus sequence on both the sides for ZFN-evoked homology directed repair. Both the CCR5 homology arms were PCR amplified using specific primers and cloned in pNTKV vector using restriction enzymes AscI and HpaI (750 bp left homology arm) and HpaI and AscI (750 bp right homology arm). The final plasm ids were named as pPBPL-Oct4/Sox2/eGFP and pPBPL-Oct4/Sox2/eGFP/Puro^{R}. Construction of ZFNs, obtained by fusing 4-finger ZFPs with FokI nuclease domain variant pair, REL DKK, is described, for example in reference (10a).

### Cell Culture

Human lung fibroblast IMR90 cells were obtained from ATCC ND cultured in Minimum essential medium (Quality Biological Inc) supplemented with 10% heat-inactivated fetal bovine serum (Invitrogen), 0.1 mM non-essential amino acids (Invitrogen) and 1.0 mM sodium pyruvate (Invitrogen). The hiPSCs were maintained on irradiated mouse embryonic fibroblasts (iMEF) (R&D Systems) in DMEM/F12 culture medium supplemented with 20% knock-out serum replacer, 0.1 mM non-essential amino acids, 1 mM L-glutamine, 0.1 mM β-mercaptoethanol and 100 ng/ml human basic fibroblast growth factor (bFGF) (all from Invitrogen).

### Plasmid Transfection and Reprogramming

IMR90 cells were seeded 3×10⁵ cells per a well of 6-well plates (day 0) and maintained with IMR90 growth medium. On day 1, cells were co-transfected with plasmids of both ZFNs (pPBPL-REL and pPBPL-DKK) and donor plasmid (either pPBPL-Oct4/Sox2/eGFP or pPBPL-Oct4/Sox2/eGFP/Puro^{R}) were introduced with 1:3 ratio of TransIT transfection reagent (Mirus). The transfection was repeated on day 3 and day 5 using the same plasmids as described above. On day 7, cells were digested off the culture plate with 0.05% trypsin-EDTA (Invitrogen). Cells were then transferred onto irradiated mouse embryonic fibroblast (iMEF) feeder layer in a gelatin coated 6-well plate and cultured with human embryonic stem cell (hESC) growth medium. hiPSCs similar to human ES cell morphology (iPSC colonies) appeared on day 28-30 and were picked out and expanded in iMEF feeder layer condition.

Alkaline Phosphatase Staining of hiPSCs

Alkaline phosphatase staining was performed using the Vector Red Alkaline phophatase substrate kit I (Vector Laboratories) following the manufacturer's instruction. Briefly, cells were fixed with 2% formaldehyde for 30 minutes at room temperature and the colonies were stained with Vector Red substrate working solution for 30 minutes at RT. After 30 minutes, colonies wells were rinsed with 100 mM Tris-Cl (pH 8.0) for twice and further rinsed with double distilled water. Cells were viewed under bright field and fluorescence microscope.

### Western Blot Analysis of CFTR Expression in CCR5-Disrupted-hiPSC Lines

CCR5-CFTR hiPSCs grown in matrigel 6-well plates were lysed by NP-40 lysis buffer (50 mM Tris-HCl pH 8.0, 150 mM NaCl, 1% NP-40, PMSF 50 μl/ml). 50 μg of total protein of cell extracts were separated by 8% SDS-PAGE and transferred to PVDF membrane (Bio-rad Laboratories). The blot was blocked with TBS-Tween (0.1% Tween-20 in TBS) supplemented with 5% low fat milk powder (w/v) for 1 hr at room temperature and incubated with a Ab217 primary antibody (1:2000) (UNC Cystic Fibrosis Center). After washing the primary antibody the membrane was incubated with Peroxidase-AffiniPure Goat Anti-Mouse IgG (H+L) secondary antibody (1:2000) (Jackson ImmunoResearch) for 1 hr at room temperature. Membrane was washed again with TBS-Tween (0.1%) and developed using an ECL chemiluminescence detection system (Amersham Biosciences, NJ) according to manufacture's instructions.

### Teratoma Assays

The teratoma assays using CFTR expressing CCR5-heterozygous hiPSCs are being conducted by the commercial firm, Applied Stem Cells, Inc., as a fee-for-service.

### Example 2

### Introduction

As discussed below, we have successfully generated both CCR5-modified heterozygous hiPSCs and biallele CCR5-mutant hiPSCs by targeted addition of stem cell factor genes at the CCR5 locus using ZFN technology. This makes CCR5 gene a desired “safe-harbor” locus within the human genome for targeted insertion of the stem cell factors to generate human induced pluripotent stem cells (hiPSC), followed by the removal of the stem cell factor genes from CCR5-heterozygous hiPSCs by CRE/loxP-mediated excision.

### Results

Efficient Generation of Human Induced Pluripotent Stem Cells (hiPSC) by Targeted Insertion Of Oct4/Sox2 Genes at the CCR5 Locus of Human Lung Fibroblasts, Using Designed ZFNs

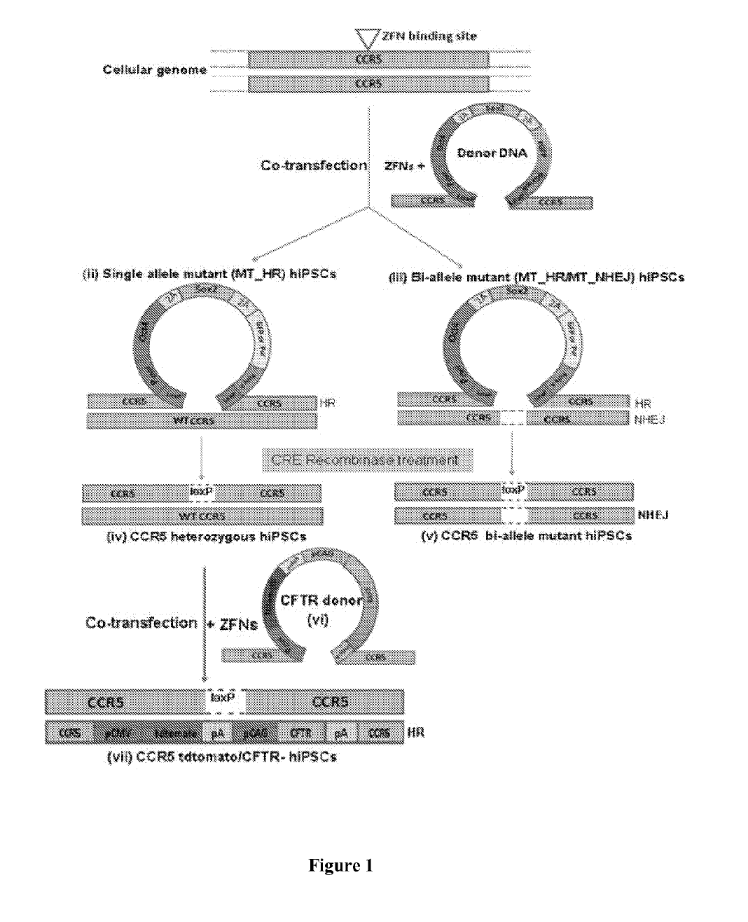

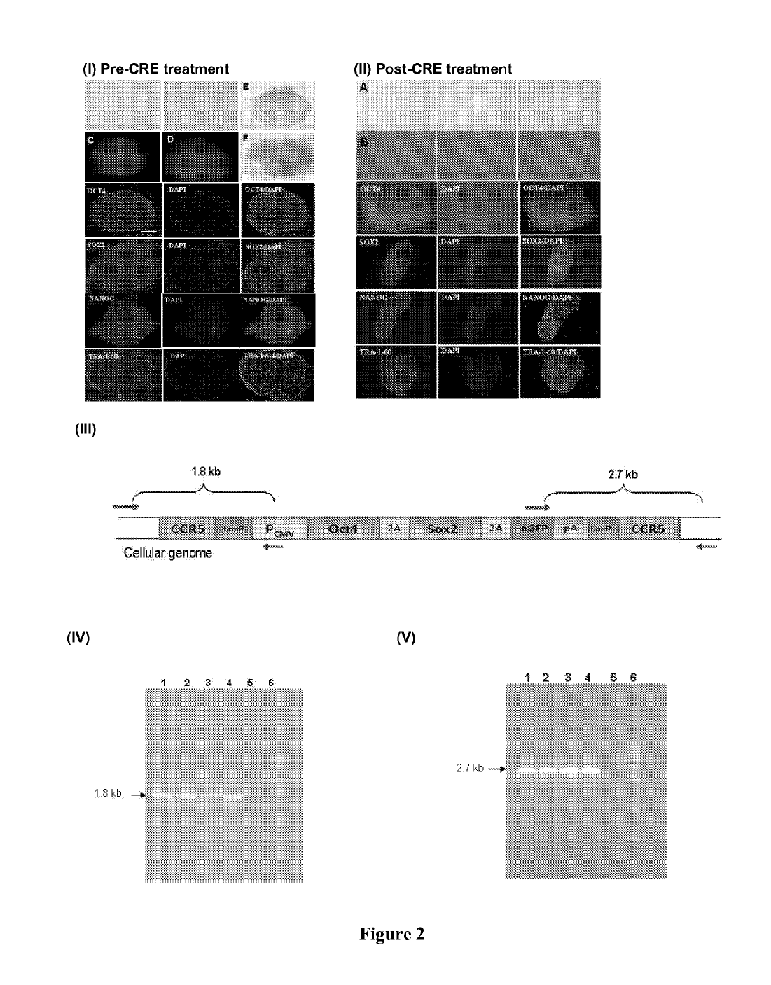

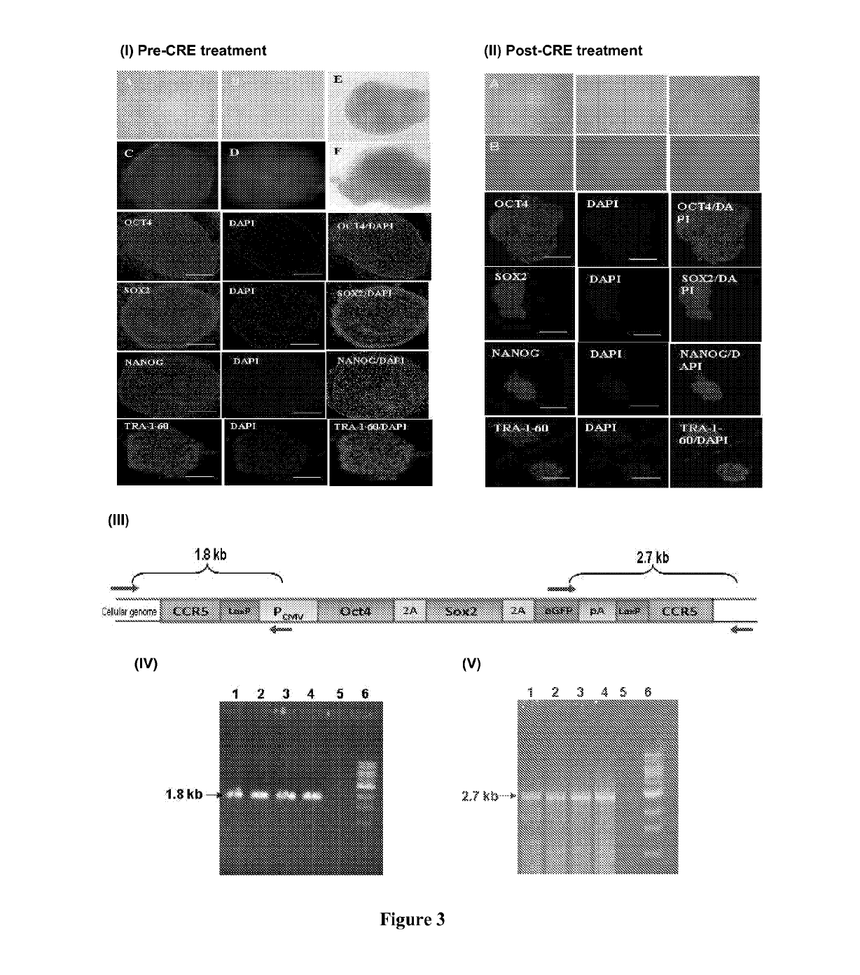

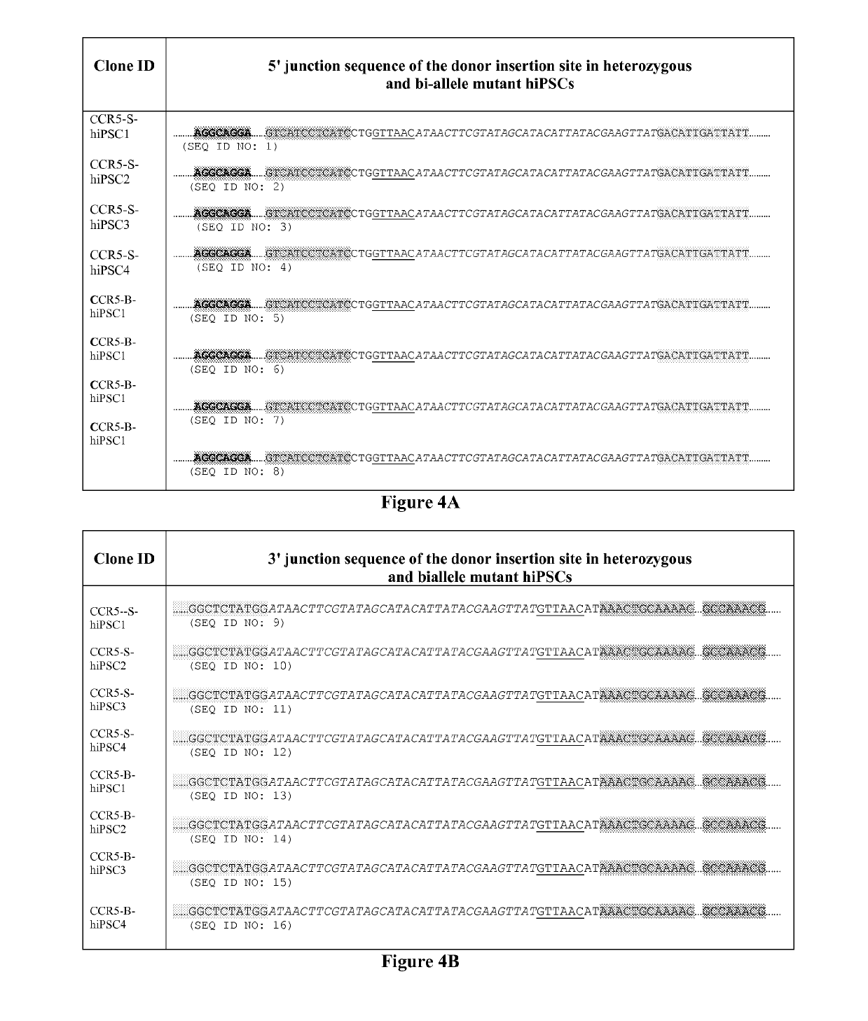

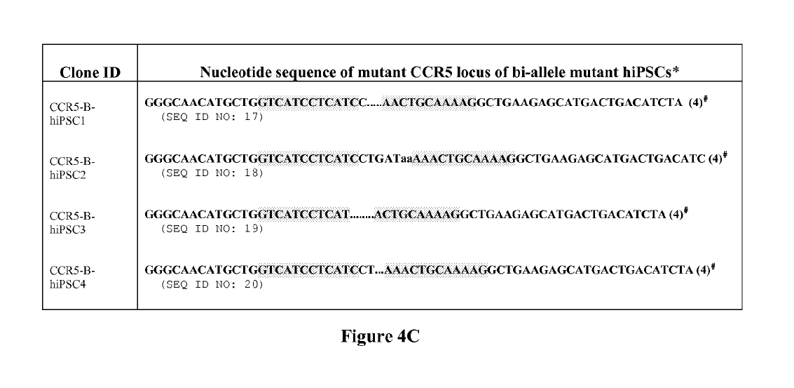

We have achieved efficient generation of hiPSCs by precisely targeted insertion of the pluripotency genes (Oct4/Sox2/GFP or Puro) flanked by loxP sites, at the CCR5 locus of human lung fibroblasts (IMR90 cells), using ZFN-mediated gene targeting in combination with small molecule HDAC inhibitor, VPA (FIG. 1, FIG. 2 & FIG. 3). The donor plasmid contained a single polycistronic cassette that would express Oct4, Sox2 and GFP (and/or Puro), each separated by 2A peptides, from the P_{cmv} promoter flanked by CCR5 homology (FIG. 1) for targeted insertion at the CCR5 locus of human fibroblasts. This novel approach for efficient generation of precisely targeted genetically well-defined hiPSCs circumvents many of the problems associated with current methods that use viral vectors to deliver the pluipotency genes to somatic cells. By this strategy, we have achieved not only targeted disruption of the CCR5 gene locus, but at the same time we have enabled efficient induction and generation of hiPSCs from somatic cells. The CCR5-gene-disrupted hiPSCs were characterized for their pluripotency markers [FIG. 2 (I) & FIG. 3(I)] before CRE treatment. As expected, all the pluripotency markers were expressed in all the single colony clones that were examined. PCR analysis of the 5′ and 3′ junctions of the donor insertion sites using appropriate primers (one primer outside the CCR5 homology arms of the donor and the other anchored within the donor) as shown FIG. 2 (III) for heterozygous hiPSCs and as shown in FIG. 3 (III) for CCR5 biallele mutant hiPSCs, gave the expected size bands of 1.8 kb and 2.7 kb, respectively. The PCR fragments were then cloned and sequenced to determine the nucleotide sequence at 5′ and 3′ junctions of the insertion sites of the hiPSCs, which revealed the presence of both CCR5 single allele mutant (MT_HR) hiPSCs resulting from HR (FIG. 2; FIG. 4A and Table 1A & FIG. 4B and Table 1B) and CCR5 bi-allelic mutant (MT_HR/MT_NHEJ) hiPSCs resulting from HR and NHEJ, respectively, which are shown in FIG. 3 and FIG. 4 (A, B & C) and Table 1 (A, B & C).

TABLE 1A
Sequence analysis of 5′ junction of the donor insertion sites of heterozygous
CCR5-mutant hiPSCs and biallele mutant hiPSCs, before CRE treatment

| | 5′ junction sequence of the donor insertion site in heterozygous |
|---|---|
| Clone ID | and bi-allele mutant hiPSCs |
| CCR5-S- | 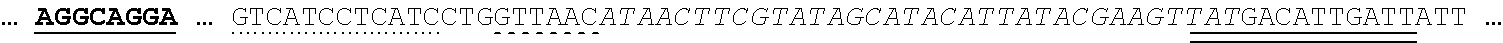 |
| hiPSC1 | (SEQ ID NO: 1) |
| CCR5-S- | 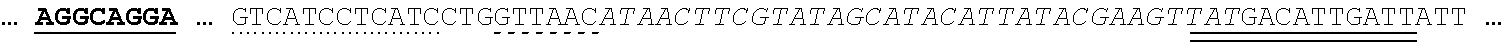 |
| hiPSC2 | (SEQ ID NO: 2) |
| CCR5-S- | 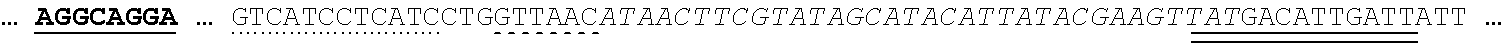 |
| hiPSC3 | (SEQ ID NO: 3) |
| CCR5-S- | 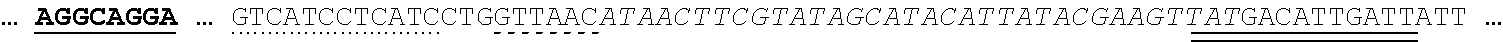 |
| hiPSC4 | (SEQ ID NO: 4) |
| CCR5-B- | 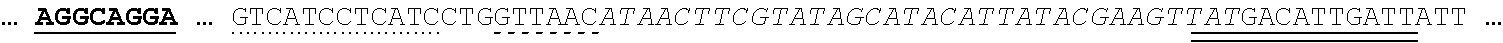 |
| hiPSC1 | (SEQ ID NO: 5) |
| CCR5-B- | 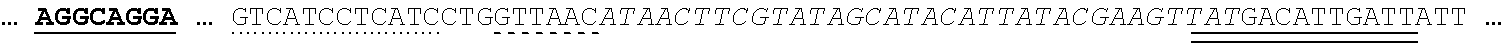 |
| hiPSC1 | (SEQ ID NO: 6) |
| CCR5-B- | 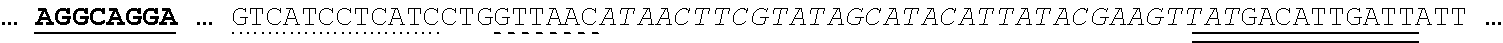 |
| hiPSC1 | (SEQ ID NO: 7) |
| CCR5-B- | 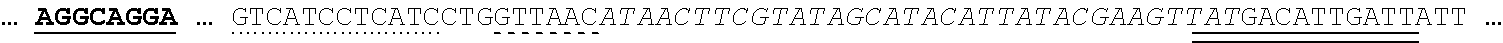 |
| hiPSC1 | (SEQ ID NO: 8) |

*The other CCR5 allele in these hiPSCs is wild type. Endogenous CCR5 sequence outside the donor homology arm is underlined, ZFN target site present in hCCR5 gene is dotted underlined. HpaI site is dashed underlined. LoxP site is in italics. CMV promoter sequence is double underlined. PCR fragments were amplified from each of the single allele mutant hiPSC clones using primers listed in Supplementary table were subcloned into E. coli and sequenced. The endogenous CCR5 and transgene junction sequence is shown in table. S, single allele (heterozygous) CCR5 mutant hiPSCs; B, biallele CCR5 mutant hiPSCs. (See FIG. 4A).

TABLE 1B
Sequence analysis of 3′ junction of the donor insertion sites in heterozygous
CCR5-mutant hiPSCs and biallele mutant hiPSCs, before CRE treatment

| Clone | 3′ junction sequence of the donor insertion site in heterozygous |
|---|---|
| ID | and biallele mutant hiPSCs |
| CCR5--S- | 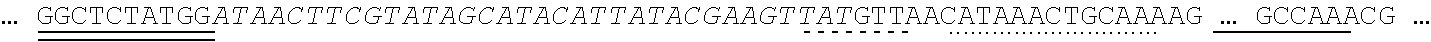 |
| hiPSC1 | (SEQ ID NO: 9) |
| CCR5-S- | 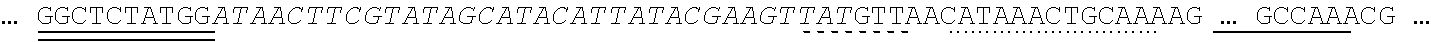 |
| hiPSC2 | (SEQ ID NO: 10) |
| CCR5-S- | 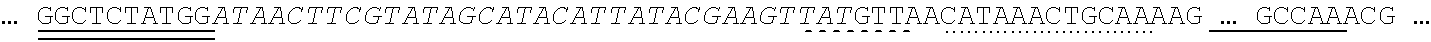 |
| hiPSC3 | (SEQ ID NO: 11) |
| CCR5-S- | 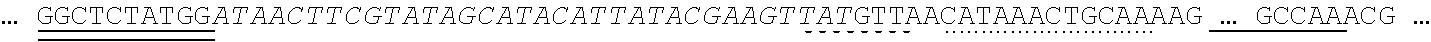 |
| hiPSC4 | (SEQ ID NO: 12) |
| CCR5-B- | 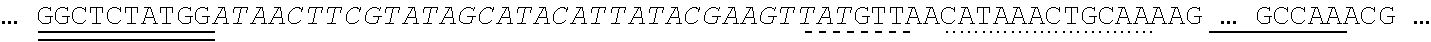 |
| hiPSC1 | (SEQ ID NO: 13) |
| CCR5-B- | 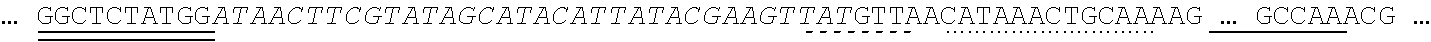 |
| hiPSC2 | (SEQ ID NO: 14) |
| CCR5-B- | 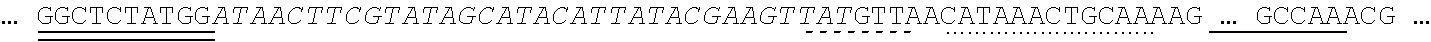 |
| hiPSC3 | (SEQ ID NO: 15) |
| CCR5-B- | 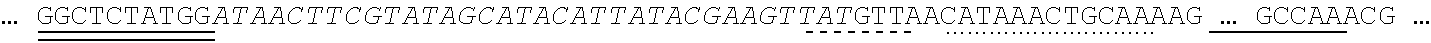 |
| hiPSC4 | (SEQ ID NO: 16) |

*The other CCR5 allele in these hiPSCs is wild type. pA terminator sequence is double underlined. LoxP site is shown in italics. HpaI site is dashed underlined. ZFN target site present in hCCR5 gene is dotted underlined. Endogenous CCR5 sequence is single underlined. S, single allele (heterozygous) CCR5 mutant hiPSCs; B, biallele CCR5 mutant hiPSCs. (See FIG. 4B).

TABLE 1C
Analysis of the CCR5 locus repaired by NHEJ in the biallele mutant hiPSCs,
before CRE treatment

| Clone ID | Nucleotide sequence of mutant CCR5 locus of bi-allele mutant hiPSCs* |
|---|---|
| CCR5-B- | GGGCAACATGCTGGTCATCCTCATCC.....AACTGCAAAAGGCTGAAGAGCATGACTGACATCTA (4)^{#} |
| hiPSC1 | (SEQ ID NO: 17) |
| CCR5-B- | GGGCAACATGCTGGTCATCCTCATCCTGATaaAAACTGCAAAAGGCTGAAGAGCATGACTGACATC |
| hiPSC2 | (4)^{#} |
| | (SEQ ID NO: 18) |
| CCR5-B- | GGGCAACATGCTGGTCATCCTCAT........ACTGCAAAAGGCTGAAGAGCATGACTGACATCTA (4)^{#} |
| hiPSC3 | (SEQ ID NO: 19) |
| CCR5-B- | GGGCAACATGCTGGTCATCCTCATCCT...AAACTGCAAAAGGCTGAAGAGCATGACTGACATCTA (4)^{#} |
| hiPSC4 | (SEQ ID NO: 20) |

*PCR fragments amplified from each of the bi-allele hiPSC clones were subcloned into E. coli. Four recombinant clones from each experiment were sequenced. The number of times the same sequence appeared is shown in brackets. B, biallele CCR5 mutant hiPSCs. (See FIG. 4C).
^{#}The other CCR5 allele in these hiPSCs is mutated by insertion of the stem cell factor genes (Tables 2A & 2B). ZFN target sites present in hCCR5 gene are double underlined.

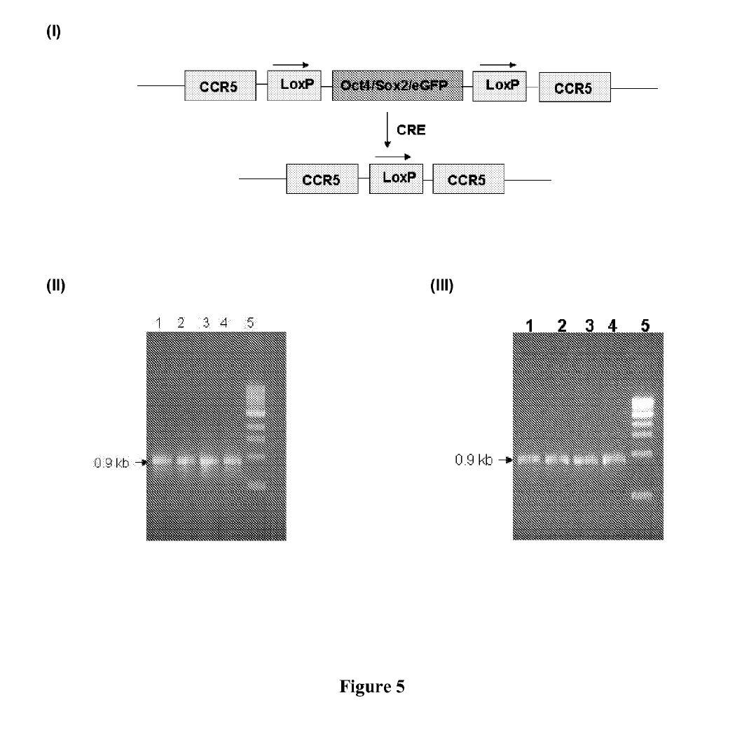

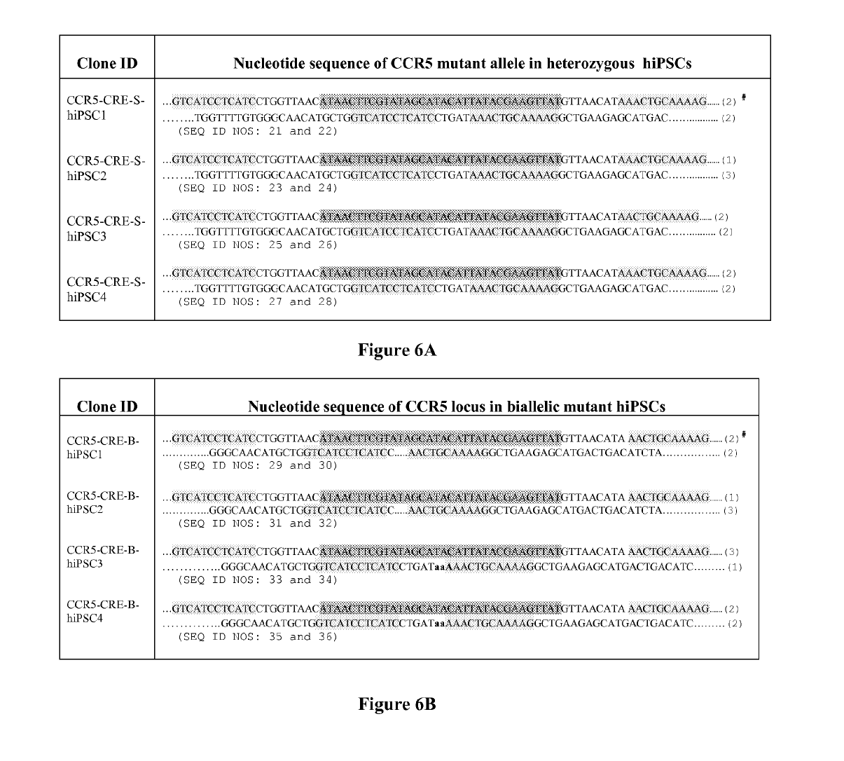

The heterozygous hiPSCs and biallele CCR5-disrupted hiPSCs were then treated with Cre recombinase to remove the stem cell factor genes (Oct4/Sox2/GFP or Puro) [FIG. 5(I)]. The heterozygous hiPSCs and biallele CCR5-disrupted hiPSCs were then characterized for their pluripotency markers. As expected, all the pluripotency markers were expressed in all the single colony clones that were examined [FIG. 2 (II) & FIG. 3(II)]. PCR analysis of single cell colonies of the heterozygous hiPSCs and biallele CCR5-disrupted hiPSCs were performed using similar primers flanking the CCR5-specific ZFN target sites, which respectively yielded the expected 0.9 kb size fragments. The PCR profile for heterozygous hiPSCs is shown in [FIG. 5(II)], while that of the biallele CCR5-disrupted hiPSCs are shown in FIG. 5 (III). The PCR fragments were then cloned and sequenced to determine the nucleotide sequence of the CCR5 mutations in the heterozygous hiPSCs and biallele CCR5-disrupted hiPSCs, which are shown in FIG. 6A and Table 2A and FIG. 6B and Table 2B. As expected, the CRE treated heterozygous hiPSCs has one CCR5 wild type allele and the other CCR5 allele disrupted by a loxP site; the CRE treated biallele CCR5-disrupted hiPSCs revealed one CCR5 allele disrupted NHEJ mutations as prior to CRE treatment and the other CCR5 allele disrupted with a loxP site. Insertion of loxP site, like the NHEJ mutations, result in functional deletion of CCR5 in the biallele CCR5-mutated hiPSCs.

TABLE 2A
Sequence analysis of the CCR5 mutant allele in heterozygous hiPSCs, after CRE
treatment

| Clone ID | Nucleotide sequence of CCR5 mutant allele in heterozygous hiPSCs |
|---|---|
| CCR5-CRE- | . . . GTCATCCTCATCCTGGTTAACATAACTTCGTATAGCATACATTATACGAAGTTATGTTAACATAAACTGCAAAAG |
| S-hiPSC1 | . . . (2) # |
| | . . . TGGTTTTGTGGGCAACATGCTGGTCATCCTCATCCTGATAAACTGCAAAAGGCTGAAGAGCATGAC . . . (2) |
| | (SEQ ID NOS: 21 and 22) |
| CCR5-CRE- | . . . GTCATCCTCATCCTGGTTAACATAACTTCGTATAGCATACATTATACGAAGTTATGTTAACATAAACTGCGAAAAG |
| S-hiPSC2 | . . . (1) |
| | . . . TGGTTTTGTGGGCAACATGCTGGTCATCCTCATCCTGATAAACTGCAAAAGGCTGAAGAGCATGAC . . . (3) |
| | (SEQ ID NOS: 23 and 24) |
| CCR5-CRE- | . . . GTCATCCTCATCCTGGTTAACATAACTTCGTATAGCATACATTATACGAAGTTATGTTAACATAACTGCAAAAG |
| S-hiPSC3 | . . . (2) |
| | . . . TGGTTTTGTGGGCAACATGCTGGTCATCCTCATCCTGATAAACTGCAAAAGGCTGAAGAGCATGAC . . . (2) |
| | (SEQ ID NOS: 25 and 26) |
| CCR5-CRE- | . . . GTCATCCTCATCCTGGTTAACATAACTTCGTATAGCATACATTATACGAAGTTATGTTAACATAAACTGCAAAAG |
| S-hiPSC4 | . . . (2) |
| | . . . TGGTTTTGTGGGCAACATGCTGGTCATCCTCATCCTGATAAACTGCAAAAGGCTGAAGAGCATGAC . . . (2) |
| | (SEQ ID NOS: 27 and 28) |

*The other CCR5 allele (bottom strand of each of hiPSCs) is wild type. ZFN target sites present in hCCR5 gene are double underlined. LoxP site is single underlined.
# PCR fragments amplified from each of the heterozygous hiPSC clones (after CRE treatment) were subcloned into E. coli. Four recombinant clones from each experiment were sequenced. The number of times the same sequence appeared is shown in brackets. S, single allele (heterozygous) CCR5 mutant hiPSCs. (See Fig. 6A).

TABLE 2B
Sequence analysis of the CCR5 mutant alleles in the bi-allele mutant hiPSCs,
after CRE treatment

| Clone ID | Nucleotide sequence of CCR5 locus in biallelic mutant hiPSCs |
|---|---|
| CCR5-CRE-B- | . . . GTCATCCTCATCCTGGTTAACATAACTTCGTATAGCATACATTATACGAAGTTATGTTAACATA |
| hiPSC1 | AACTGCAAAAG . . . (2)^{#} |
| | . . . GGGCAACATGCTGGTCATCCTCATCC . . . AACTGCAAAAGGCTGAAGAGCATGACTGACATCTA . . . (2) |
| | (SEQ ID NOS: 29 and 30) |
| CCR5-CRE-B- | . . . GTCATCCTCATCCTGGTTAACATAACTTCGTATAGCATACATTATACGAAGTTATGTTAACATA |
| hiPSC2 | AACTGCAAAAG . . . (1) |
| | . . . GGGCAACATGCTGGTCATCCTCATCC . . . AACTGCAAAAGGCTGAAGAGCATGACTGACATCTA . . . (3) |
| | (SEQ ID NOS: 31 and 32) |
| CCR5-CRE-B- | . . . GTCATCCTCATCCTGGTTAACATAACTTCGTATAGCATACATTATACGAAGTTATGTTAACATA |
| hiPSC3 | AACTGCAAAAG . . . (3) |
| | . . . GGGCAACATGCTGGTCATCCTCATCCTGATaaAAACTGCAAAAGGCTGAAGAGCATGACTGACATC . . . (1) |
| | (SEQ ID NOS: 33 and 34) |
| CCR5-CRE-B- | . . . GTCATCCTCATCCTGGTTAACATAACTTCGTATAGCATACATTATACGAAGTTATGTTAACATA |
| hiPSC4 | AACTGCAAAAG . . . (2) |
| | . . . GGGCAACATGCTGGTCATCCTCATCCTGATaaAAACTGCAAAAGGCTGAAGAGCATGACTGACATC . . . (2) |
| | (SEQ ID NOS: 35 and 36) |

ZFN target sites present in hCCR5 gene are double underlined. LoxP site is single underlined.
^{#} PCR fragments amplified from each of the biallele mutant hiPSC clones were subcloned into E. coli. Four recombinant clones from each experiment were sequenced. The number of times the same sequence appeared is shown in brackets. Insertions are shown in bold lowercase letters. Dots denote deletions. B, biallele CCR5 mutant hiPSCs. (See FIG 6B).

### Discussion

In these example, we have attempted to address two important questions in proof-of-principle experiments using human lung fibroblasts (IMR90 cells): (1) Could ZFN-mediated gene targeting, be used for efficient generation of hiPSCs by targeted addition of pluripotency genes to one of the CCR5 alleles in human cells? and (2) Could the functionality of the reprogrammed hiPSCs, after the removal of the pluripotency genes by CRE treatment, be reshaped by targeted addition of a therapeutic gene for functional complementation at the remaining wild type CCR5 allele? The answer to both questions appears to be yes.

Generation of hiPSCs from Human Lung Fibroblasts by Targeted Addition of Pluripotency Genes at the CCR5 Locus of the Human Genome

To our knowledge, reprogramming of somatic cells or primary cells to hiPSCs using designed ZFNs, has not been reported previously. Generation of hiPSCs using site-specific integration with phage integrase (ΦC31) has been reported elsewhere, for example reference (29). Although the derived hiPSCs had only a single integration in each line, the locations of integration were random in different lines, favoring intergenic regions. It is quite possible that site-specific integration using ΦC31 integrase could result in insertions at critical genes or control regions in some cell lines to disrupt the normal function of the cells. In our experiments using designed ZFNs, we observed integration of the pluripotency genes only at the CCR5 locus in all the single colony hiPSCs that we examined. Thus, unlike ΦC31, ZFN-mediated gene targeting results in uniform highly specific integration of the pluripotency genes at the desired CCR5 locus of the lung fibroblasts while reprogramming to hiPSCs.

Genetic engineering of hiPSCs using designed ZFNs or TALE nucleases (TALENs) have been reported previously in literature (20-25); however, the hiPSCs used in these studies were generated by using standard viral vector reprogramming methods, which employs random integrations of pluripotency genes within the human genome (30) unlike our novel approach which uses targeted addition to a specific site (CCR5 locus) to generate hiPSCs using designed ZFNs. The “safe harbor” AAVS1 locus of the human genome has been previously used for targeted addition of transgenes in hiPSCs using designed ZFNs (21-24). Thus, at least two safe harbor loci (CCR5 and AAVS1) within the human genome, are currently available for targeted addition of multiple transgenes for ectopic expression in human cells. Alternatively, in our approach, one could use targeted addition of pluripotency genes to the AAVS1 locus to reprogram human cells into hiPSCs and use the CCR5 locus for targeted addition of therapeutic transgenes for functional complementation in cells with corresponding gene defects or vice versa.

### Summary

Human induced pluripotent stem cells (hiPSCs) were generated from human lung fibroblasts (IMR90 cells) by site-specific insertion of Oct4 and Sox2 transcription factor genes flanked by LoxP sites to the CCR5 locus to form both heterozygous and bi-allele CCR5-disrupted hiPSCs. The Oct4 and Sox2 stem cell factors in conjunction with valproic acid (VPA) induced efficient reprogramming of lung fibroblasts to CCR5-modified hiPSCs. Subsequent treatment of hiPSCs with Cre, resulted in the removal of the stem cell factor genes from both the CCR5-heterozygous hiPSCs and the bi-allele CCR5-disrupted hiPSCs.

### Example 3

### Targeted Addition of the CFTR Transcription Unit to the CCR5 Safe Harbor Locus of hiPSCs Using Designed Zinc Finger Nucleases

The ZFN-mediated gene targeting was used for site-specific addition and expression of the large CFTR transcription unit in human cells, namely at: (1) The transduced CCR5 locus of model HEK293 Flp-In cells; and (2) The endogenous locus of human induced pluripotent stem cells (hiPSCs). Site-specific addition and expression of the CFTR transcription unit at the remaining CCR5 wild-type allele of the CCR5-heterozygous hiPSCs, was achieved using designed ZFNs and a donor containing the large CFTR transcription unit flanked by CCR5 homology arms. CFTR is expressed efficiently from the endogenous CCR5 locus in these hiPSCs.

### Introduction

Cystic fibrosis (CF) is a fatal autosomal recessive genetic disease affecting about 1 in 2,000 Caucasians and 1 in 17,000 African-americans (1). The pathology of the disease is in the dysfunction of the CFTR chloride (Cl⁻) channels in epithelial cells. In CF patients, epithelial cells lining the respiratory, GI and exocrine systems even though express Cl⁻ channels on the cell surface to a certain extent, the channels, however, cannot be activated to open via normal regulatory pathways. In normal cells, a regulatory factor, cAMP, stimulates the opening of the Cl⁻ channels, but cAMP fails to do so in CF cells. This abnormality disrupts the ion permeability and electric potential of the epithelial cells. Since Cl⁻ ion movement is paired with the movement of Na+ in through the epithelial cells, the failure of Cl⁻ channels produces an imbalance in both Cl⁻ secretion and Na+ ion absorption. Therefore, the ion concentration gradient is disrupted, interfering with the process of osmosis, producing a dehydrated and highly viscous mucous that is characteristic of the clinical disease. The affected organs include the lungs, intestine, pancreas and sweat ducts, but death results from respiratory problems. The gene responsible for CF, the cystic fibrosis transmembrane conductance regulator (CFTR) encodes a cAMP-regulated Cl⁻ channel, which spans the apical membrane of epithelial cells (2,3). Although CF is caused by a variety of mutations in the CFTR gene, the most common mutation, which accounts for about 70% of all mutant CF cells, is the deletion of three base pairs within exon 10 of the CFTR gene. This results in the removal of phenylalanine 508, from the first nucleotide-binding domain of the protein. The ΔF508 mutation can be viewed primarily as a protein trafficking defect since it is incorrectly processed and most of it becomes trapped in the endoplasmic reticulum. The defective sub-cellular localization of the CFTRΔF508 protein can be corrected in two ways: (i) High-level expression of ΔF508 protein in heterologous cells, allows some CFTR to reach the plasma membrane (4). (ii) Trafficking defect was shown to be temperature dependent such that incubation of fibroblasts expressing the ΔF508 protein below 30° C. rather than 37° C. generated a cAMP-activated Cl⁻ channel. ΔF508 protein appears to show temperature sensitivity to protein folding defect. The generation of a ΔF508 CF mouse and pig models that have the same CFTR protein trafficking defect as that present in 90% of CF patients has also been reported (3).

Identification of the CFTR gene has allowed research into treatment that enables the defective gene to be replaced. Because CF is a recessive disease, only one copy of the normal gene is required to correct the physiological fault in the target cells. Two approaches could be used to correct the defect. In germ-line therapy, the fetus is altered during the early stages of mitosis after egg fertilization so that all the daughter cells are genotypically normal. The other alternative is somatic cell gene therapy. Here, the complementary DNA, which carries the corrected form of the CFTR gene, is spliced into the DNA of a disabled adenovirus. The recombinant virus is then delivered into the lung epithelia by inhalation via a spray or nebulizer (2). Once in the lungs, the virus enters into the cell nucleus and a normal CFTR protein is manufactured, supplementing the function of the native DNA. The success of this approach depends on the efficient transfer of cDNA into the cells. Problems associated with this approach are as follows: (i) Expression of cDNA, delivered by the adenovirus, is short-lived and repeated administration of high doses only partially corrected the CFTR defect in Cl⁻ transport in vivo and had no effect in Na⁺ transport (5). (ii) Problems also arise from the viral properties of the adenovirus due to humoral response against viral cell wall. In order for this approach to be successful gene therapy has to deliver CFTR DNA to about 5-10% of lung epithelial cells without initiating a toxic humoral response within the lungs (6). The use of embryonic and adult stem cells for lung repair and regeneration after injury has been proposed a potential therapeutic approach for CF and other lung diseases (7).

### Results

### Site-Specific Integration of Wild-Type CFTR and CFTRΔ508F Transcription Units, Respectively, at the Transduced CCR5 Locus in Model HEK293 Flp-in Cells, Using Designed ZFNs

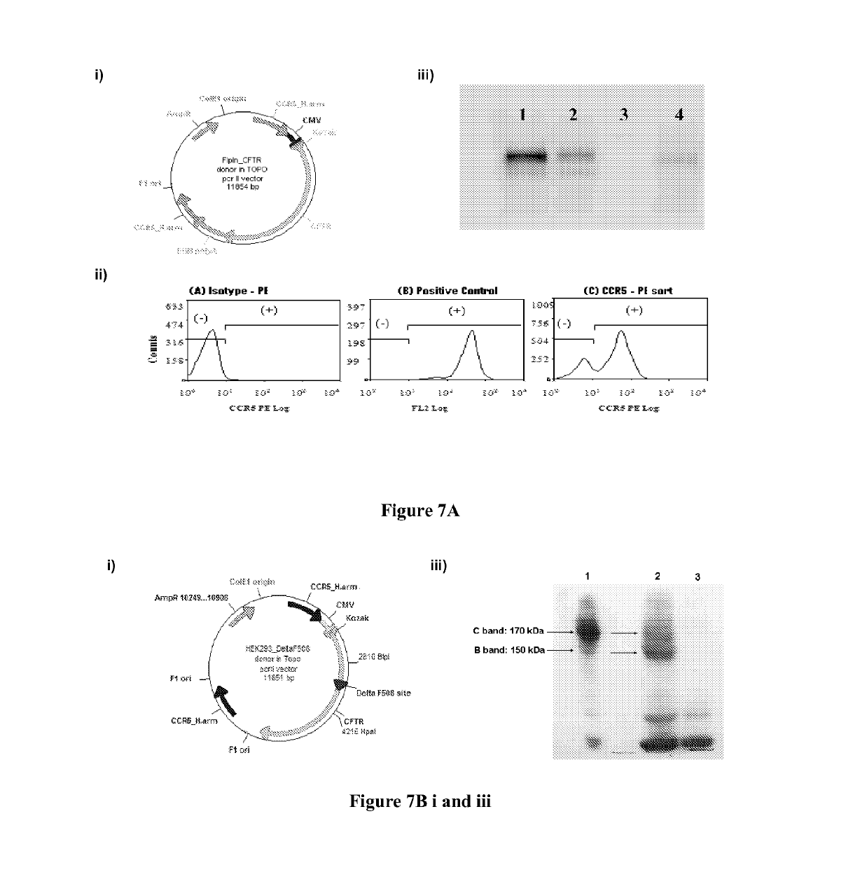

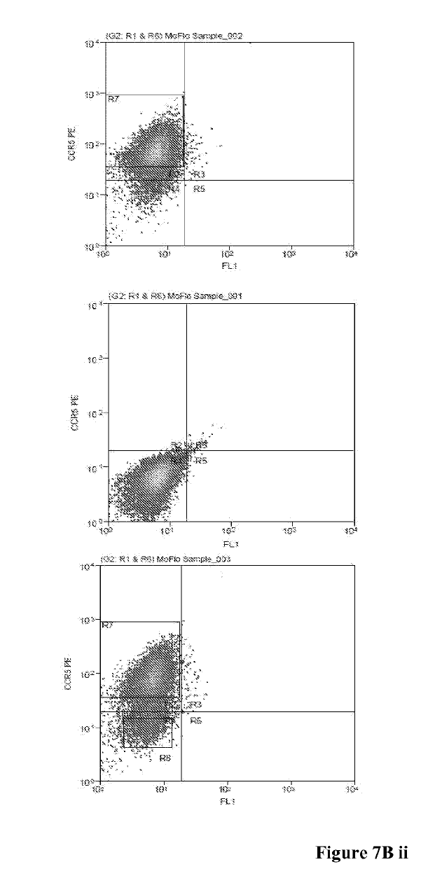

The donor plasmid substrates contained either WT CFTR cDNA or mutant CFTRΔ508F cDNA with appropriate transcription and translational signals (P_{cmv} promoter and BGH polyA tail) for gene expression flanked by ˜700-800 bp CCR5 homology arms on either side to promote recombination at the targeted CCR5 chromosomal locus in human cells [FIG. 7A (i) & FIG. 7B (i)]. The designed 4-finger ZFNs along with CFTR donor DNA (or CFTRΔ508F cDNA) were transfected into the CCR5-expressing HEK293 Flp-In cells, which were previously generated by inserting CCR5 cDNA at the FRT site (18). The cells were then analyzed for CCR5 expression after 7 days post-transfection by FACS. About 23% of cells lost CCR5 expression in the case of wild-type CFTR donor [FIG. 7A (ii)], while ˜17% became CCR5 negative with mutant CFTRΔ508F donor [FIG. 7B (ii)]. FACS sorting was used to isolate CCR5-modified single HEK293 Flp-In cell clones into 96-well plates and the single cell clones were cultured for several more weeks. The genomic DNA was then isolated from these clones to determine their genotype by PCR using appropriate primers. Many of these clones showed the desired genotype. DNA sequence analyses of the transduced CCR5 locus further confirmed the genotype of these clones.

Western blot profile of the CFTR expression in two representative CCR5-gene-modified single colonies and the previously reported T84 cell line are shown in FIG. 7A (iii). The Western blot was probed using the antibody Ab217, anti C-terminal monoclonal mouse antibody against CFTR, which was purchased from UNC Center for Cystic Fibrosis Research. The CFTR expression by targeted introduction of CFTR cDNA at the transduced CCR5 locus in HEK293 cells appears to be ˜5-20-fold higher than in a commercially available T84 cell line, which was generated using traditional approaches. The Western blot profile of the mutant CFTRΔ508F expression of the CCR5-gene-modified single clone is shown in FIG. 7B (iii).

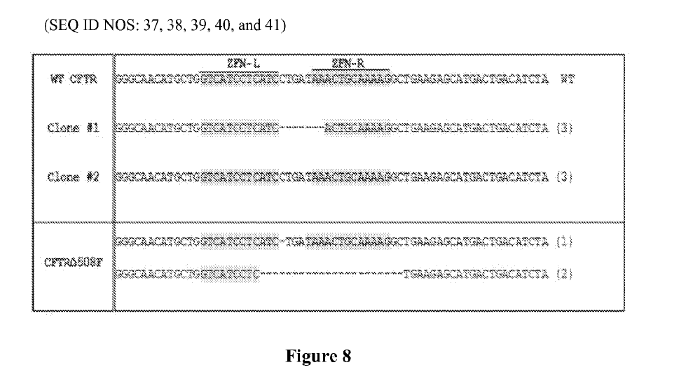

Sequence analysis of the endogenous CCR5 loci of the WT CFTR HEK293 Flp-In lines revealed that the sites were cleaved by ZFNs and then repaired by NHEJ in one of the clones, while in the other clone the sites were not cut (FIG. 8 and Table 3). Similar sequence analysis of the CFTRΔ508F HEK293 Flp-In line revealed that the endogenous CCR5 sites were cut and repaired by NHEJ, but resulting in two different types of deletions at the two sites (FIG. 8 and Table 3). Thus, targeted addition of WT CFTR cDNA or mutant CFTRΔ508F transcription units at the transduced CCR5 site of HEK293 Flp-In cells using ZFNs has resulted in the generation two important HEK293 lines that over-express WT CFTR (FIG. 7A) and CFTRΔ508F (FIG. 7B), respectively.

TABLE 3
Analysis of the endogenous CCR5 locus of HEK293 cell lines expressing the
WT_CFTR and mutant CFTRΔ508F protein, respectively.

| | |
|---|---|
| | ZFN- L ZFN-R |
| WT CFTR | GGGCAACATGCTGGTCATCCTCATCCTGATAAACTGCAAAAGGCTGAAGAGCATGACTGACATCTA WT |
| Clone #1 | GGGCAACATGCTGGTCATCCTCATC-------ACTGCAAAAGGCTGAAGAGCATGACTGACATCTA (3) |
| Clone #2 | GGGCAACATGCTGGTCATCCTCATCCTGATAAACTGCAAAAGGCTGAAGAGCATGACTGACATCTA (3) |
| CFTRΔ508F | GGGCAACATGCTGGTCATCCTCATC-TGATAAACTGCAAAAGGCTGAAGAGCATGACTGACATCTA (1) |
| | GGGCAACATGCTGGTCATCCTC----------------------TGAAGAGCATGACTGACATCTA (2) |

(SEQ ID NOS: 37, 38, 39, 40, and 41) ZFN target sites present in hCCR5 gene are highlighted.
#PCR fragments amplified from each of the biallelic mutant HEK293-Flp In clones were subcloned into E. coli. Four recombinant clones from each subcloning experiment were sequenced. The number of times the same sequence appeared is shown in brackets. Dots denote deletions. (See FIG 8).

Targeted Addition and Expression of Wild-Type CFTR Transcription Unit at the Endogenous CCR5 Locus of CCR5-Heterozygous hiPSCs Using Designed ZFNs

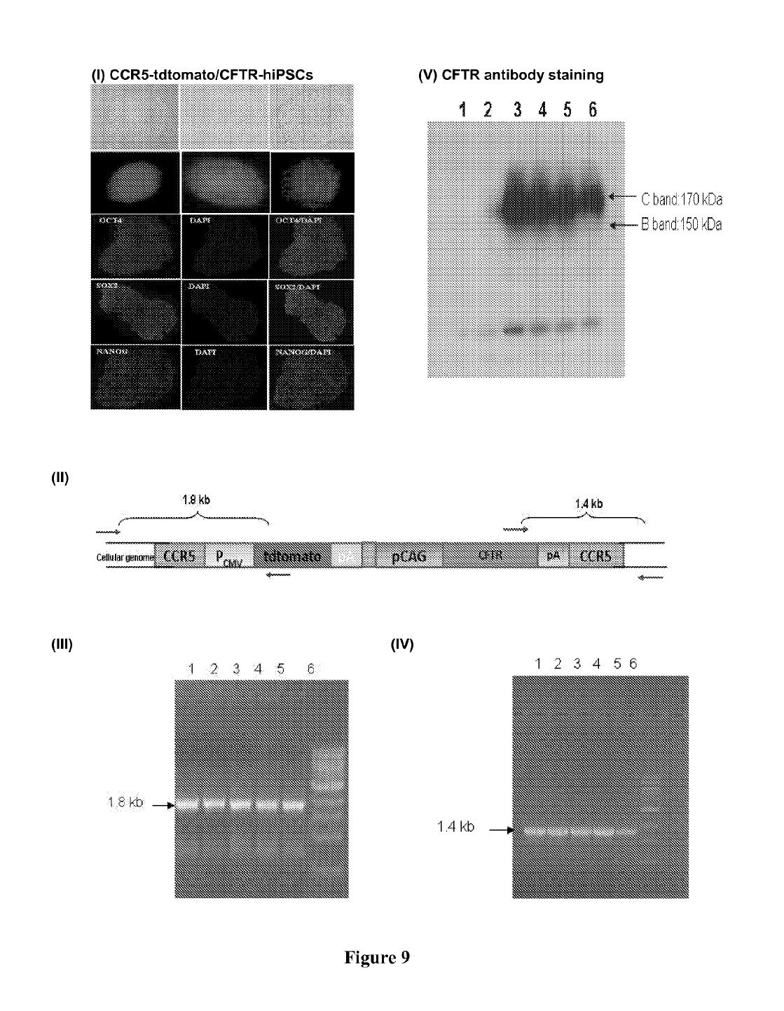

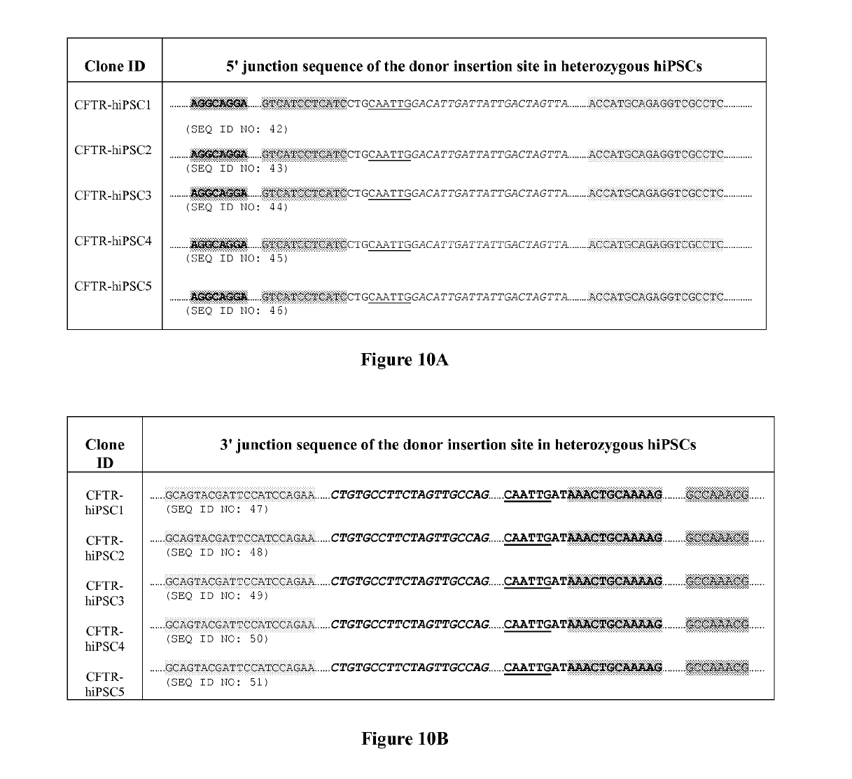

Nucleofection of CCR5-heterozygous hiPSCs using tdTomato/CFTR cDNA donor and designed ZFNs, resulted in the targeted addition of the CFTR transcription unit at the remaining wild type CCR5 allele (FIG. 9). The donor construct contained tdtomato gene under the control of the P_{cmv} promoter, while CFTR cDNA under the control of P_{cag} promoter [FIG. 9(II)]. All the pluripotency markers were expressed in all the single colony CFTR hiPSC that were examined [FIG. 9(I)]. The hiPSCs expressing tdTomato was FACS sorted and single cell colonies were isolated by serial dilution and grown [FIG. 9(I)]. As expected, all the pluripotency markers were expressed in the single colony CFTR hiPSCs that were examined [FIG. 9(I)]. PCR analysis of the 5′ and 3′ junctions of the donor insertion site were performed using appropriate primers (one primer outside the CCR5 homology arms and the other anchored within the donor) as shown in FIG. 9(II), which yielded the expected fragments for 1.8 kb and 1.4 kb, respectively. The PCR fragments were then cloned and sequenced to determine the nucleotide sequence at the 5′ and 3′ junctions of the donor insertion site, which revealed that tdTomato/CFTR donor was indeed inserted at the remaining wild type CCR5 allele of the heterozygous hiPSCs (FIG. 10 and Table 4).

TABLE 4A
Sequence analysis of 5′ junction of the tdTomato/CFTR donor insertion site in
heterozygous CCR5-mutant hiPSCs

| Clone ID | 5′ junction sequence of the donor insertion site in heterozygous hiPSCs |
|---|---|
| CFTR- | 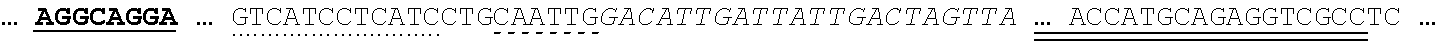 |
| hiPSC1 | (SEQ ID NO: 42) |
| CFTR- | 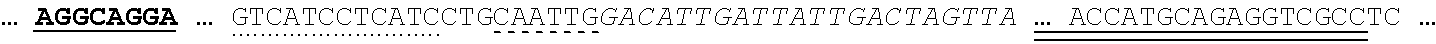 |
| hiPSC2 | (SEQ ID NO: 43) |
| CFTR- | 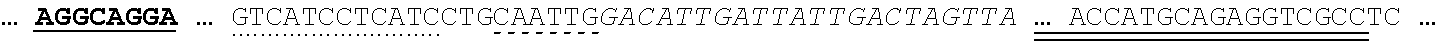 |
| hiPSC3 | (SEQ ID NO: 44) |
| CFTR- | 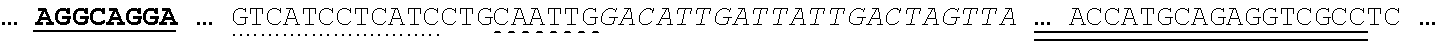 |
| hiPSC4 | (SEQ ID NO: 45) |
| CFTR- | 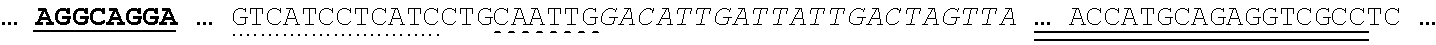 |
| hiPSC5 | (SEQ ID NO: 46) |

Endogenous CCR5 genomic sequence, outside the donor homology arm, is single underlined. ZFN target site present in hCCR5 gene is dotted underlined. MfeI site is dashed underlined. CMV promoter sequence is shown in italics. tdtomato sequence is double underlined. PCR fragments were amplified from each of the CFTR hiPSC clones using primers listed in Table 5, were subcloned into E. coli and sequenced. The endogenous genomic CCR5 and tdtomato/CFTR 5′-end junction sequence is shown above. (See FIG. 10A).

TABLE 4B
Sequence analysis of 3′ junction of the tdTomato/CFTR donor insertion site in
heterozygous CCR5-mutant hiPSCs

| Clone ID | 3′ junction sequence of the donor insertion site in heterozygous hiPSCs |
|---|---|
| CFTR- | 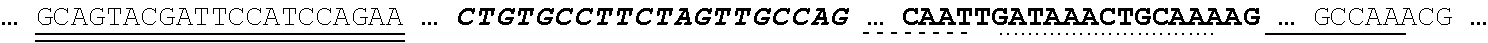 |
| hiPSC1 | (SEQ ID NO: 47) |
| CFTR- | 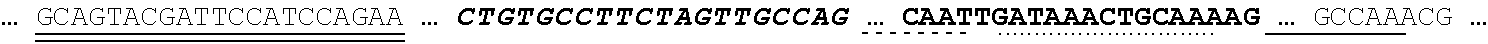 |
| hiPSC2 | (SEQ ID NO: 48) |
| CFTR- | 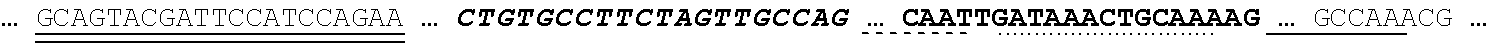 |
| hiPSC3 | (SEQ ID NO: 49) |
| CFTR- | 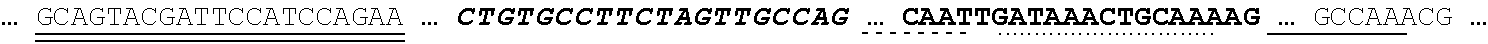 |
| hiPSC4 | (SEQ ID NO: 50) |
| CFTR- | 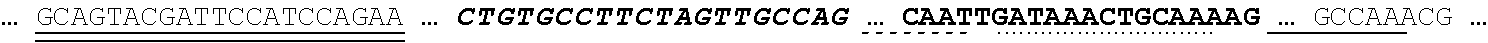 |
| hiPSC5 | (SEQ ID NO: 51) |

CFTR sequence is double underlined. pA sequence is shown in italics. Mfel site is dashed underlined. ZFN target site present in hCCR5 gene is dotted underlined. Endogenous CCR5 genomic sequence, outside the donor homology arm, is single underlined. PCR fragments were amplified from each of the CFTR hiPSC clones using primers listed in Table 5, were subcloned into E. coli and sequenced. The endogenous genomic CCR5 and tdtomato/CFTR 3′-end junction sequence is shown above. (See FIG. 10B).

TABLE 5
PCR primer sequences and amplification conditions

| Gene | | Sequence | PCR Amplification |
|---|---|---|---|
| locus | Primer | (5′ to 3′) | conditions |
| CCR5(5′end) | CCRS-F | CAACTCAAACTACAAACACAAACTTCACAG | 95° C. for 5 min, then 30 |
| | CMV-R | GGAAAGTCCCGTTGATTTTGGTGCC | cycles of 95° C. for 30 sec, |
| | | (SEQ ID NOS: 52 and 53) | 55° C. for 1 min, followed by |
| | | | extension at 72° C. for 2 min |
| CCR5(3′end) | pA-F | CTGTGCCTTCTAGTTGCCAGC | 95° C. for 5 min, then 35 |
| | CCR5-R | GAGTTTGATGCTTATTGAATGTGTAG | cycles of 95° C. for 30 sec, |
| | | (SEQ ID NOS: 54 and 55) | 52° C. for 2 min, followed by |
| | | | extension at 68° C. for 3 min |
| CCR5(NHEJ) | CCR5(endo)-F | ATGGATTATCAAGTG TCAAGTCCA | 95° C. for 5 min, then 30 |
| | CCR5(endo)-R | TCACAAGCCCACAGATATTTCC | cycles of 95° C. for 30 sec, |
| | | (SEQ ID NOS: 56 and 57) | 58° C. for 1 min, followed by |
| | | | extension at 72° C. for 5 min |
| CCR5-CFTR- | CCR5-F | GTTGCCCTAAGGATTAAATGAATGAATG | 95° C. for 5 min, then 30 |
| tdtomato | tdtomato-R | CTCCATGCGCACCTTGAAGCGCATGAAC | cycles of 95° C. for 30 sec, |
| (5′ end) | | (SEQ ID NOS: 58 and 59) | 55° C. for 1 min, followed by |
| | | | extension at 72° C. for 2 min |
| CFTR-CCR5 | CFTR-F | GCAGTACGATTCCATCCAGAAACTGCTG | 95° C. for 5 min, then 30 |
| (3′ end) | CCR5-R | GGATGAATCTTAGACCCTCTATAACAG | cycles of 95° C. for 30 sec, |
| | | (SEQ ID NOS: 60 and 61) | 55° C. for 1 min, followed by |
| | | | extension at 72° C. for 2 min |
| CCR5-CRE | CCR5-CRE-F | ACAAGATTTTATTTGGTGAGATGG | 95° C. for 5 min, then 30 |
| | CCR5-CRE-R | AGAATTGATACTGACTGTATGG | cycles of 95° C. for 30 sec, |
| | | (SEQ ID NOS: 62 and 63) | 55° C. for 30 sec, followed by |
| | | | extension at 72° C. for 1 min |

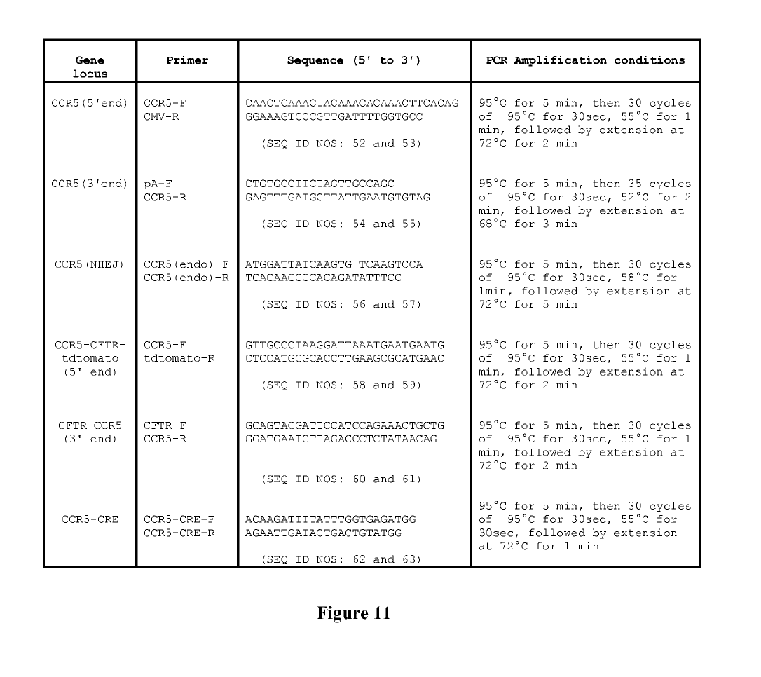

F, Forward primer and R, Reverse primer. (See FIG. 11).

### Discussion

This example shows that human CCR5 chromosomal locus can serve as a “safe harbor” for introduction of corresponding therapeutic wild type transgenes for functional complementation in various types of cells with monogenic defects. That is, the function of CCR5 is expendable, so that the gene can be cleaved and one or more transgenes of interest can be inserted and expressed ectopically to provide a functional cure for cells with monogenic defects.

Targeted Addition and Ectopic Expression of the Large CFTR Transcription Unit from the CCR5 Locus of Heterozygous hiPSCs

We have achieved site-specific addition and expression of the CFTR transcription unit at the remaining CCR5 wild-type allele of the CCR5-heterozygous hiPSCs using designed ZFNs and a donor containing the large CFTR/tdTomato transcription units flanked by CCR5 homology arms. Targeted addition of the large CFTR transcription unit to the remaining CCR5 allele of heterozygous hiPSCs was efficient and tdTomato gene expression was used as a marker gene for sorting out the CCR5-gene-modified hiPSCs by FACS. Efficient and high expression of CFTR was observed in all single cell colony hiPSCs that we examined, confirming that the CCR5 locus could serve as an ideal locus for targeted addition and ectopic expression of therapeutic transgenes for functional complementation studies in cells with corresponding gene defects, for example in cells with CF or sickle cell disease (SCD). However, how the expression of the therapeutic proteins will affect the individual cell with the corresponding gene defect is unknown at this time and remains to be studied in detail in the future.

### Summary

We have used ZFN-mediated gene targeting to insert the large WT CFTR cDNA with appropriate transcription and translation signals at a pre-determined “safe-harbor” site (the CCR5 gene locus) of the human genome under the control of a CMV/CAG promoter to achieve efficient constitutive expression of CFTR at 1) the transduced CCR5 locus in model HEK293 Flp_In cells, and 2) the endogenous CCR5 locus of the human induced pluripotent stem cells (hiPSCs).

This makes CCR5 gene a desired “safe-harbor” locus within the human genome for targeted insertion of the stem cell factors to generate human induced pluripotent stem cells (hiPSC), followed by the removal of the stem cell factor genes from CCR5-heterozygous hiPSCs by CRE/loxP-mediated excision, and then targeted addition of CFTR transcription unit at the remaining wild-type CCR5 allele for ectopic expression of the transgene. Such an approach will also permit CFTR functional complementation studies in hiPSCs with homozygous CFTRΔ508F defect or bi-allelic CFTR functional defect in the future.

### Example 4

### Targeted Addition and Expression of tdTomato/Beta-Globin Donor from the CCR5 Locus of TNC1 hiPSCs, with Homozygous Sickle Cell Disease Mutation

TNC1 line (Chou et al., 2011) was purchased from Dr. Linzhao Cheng lab (Johns Hopkins School of Medicine, Baltimore, USA). The frozen TNC1 hiPScs were thawed and cultured under MEF feeder condition. For nucleofection, TNC1 hiPSCs were passaged onto E-well matrigel plates and cultured in mTesRI (Stem cell Technologies) under feeder-free conditions. Two million TNC1 hiPSCs were digested with accutase (Sigma) for 2-3 min and neutralized by PBS. The cells were centrifuged at 100×g for 5 min, and resuspended in 100 μl of Amaxa nucleofector solution V (Amaxa Biosystems Gaithersburg Md.) with 1 μg of each pIRES vector carrying the corresponding CCR5-specific ZFNs and 8 μg of the donor plasmid containing the wild-type beta-globin and marker gene tdTomato. The whole transgene cassette was flanked by 750 bp endogeneous CCR5 locus-specific sequence on both the sides for ZFN-evoked homology directed repair. The TNC1 hiPSCs were transfected using an Amaxa nuclofector device with program A-023. 10 μM ROCK inhibitor Y 27632 was added for 1 hour prior to and immediately after nucleofection to improve the survival of dissociated hiPSCs. After nucleofection, the cells were seeded in matrigel plates and allowed to grow for at least a week before cell sorting.

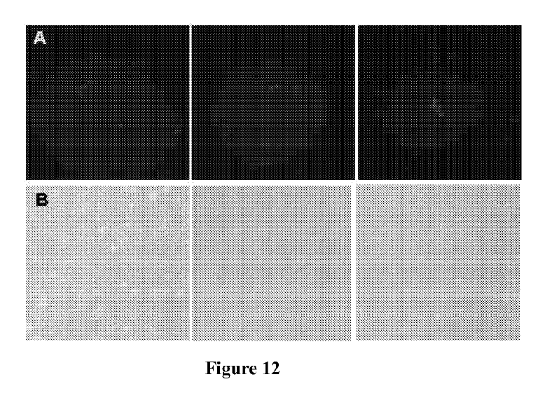

FIG. 12 shows the results of the targeted addition and ectopic expression of tdTomato/β-globin gene from the CCR5 locus of TNC1 hiPSC cell line, which contains homozygous Sickle Cell Disease mutation (SCD) for functional complementation. FIG. 12A shows tdTomato fluorescence images of CCR5-modified SCD hiPSCs and FIG. 12B shows bright field images of CCR5-modified SCD hiPSCs.

The tdTomato-expressing cells will be sorted by FACS. Serial dilution will be used to isolate single cell colonies, which will be characterized by PCR, sequencing and immunostaining as well as monitored for β-globin gene expression.

### Example 5

### CCR5-Disrupted Biallele Mutant hiPSCs

### Introduction

We chose the CCR5 gene as the safe-harbor genomic locus for the reason that no deleterious effects have been observed in individuals who carry homozygous Δ32 base pair deletion mutant alleles of the gene. CCR5 gene encodes a co-receptor on CD4+ cells and is associated with HIV-1 infection. Individuals carrying mutant CCR5 gene, who are otherwise healthy, are resistant to HIV-1 infection.

### Results

The biallele CCR5-disrupted hiPSCs can be differentiated into CD34+ haematopoietic cells and the biallelic CCR5-disrupted hiPSC-derived cells' resistance to HIV infection is being tested. The bi-allelic CCR5-disrupted hiPSCs should be permanently resistant to HIV; and they should also selectively survive and expand in NSG mice after HIV infection; they should also provide a reservoir of healthy and un-infectable cells for autologous transplantation in patients.

### Discussion

ZFN-mediated targeted cleavage of genes encoding receptors for viruses can be used to block expression of such receptors, thereby prevent viral infection and/or viral spread in a host organism. Targeted mutagenesis of genes coding viral receptors (e.g. the CCR5 and CXCR4 receptors for HIV) can be used render the receptors unable to bind to virus, thereby preventing new infection and blocking the spread of existing infections (26-28). Holt et al have previously reported high efficiency ZFN-mediated disruption of the CCR5 locus by NHEJ in human CD34+ HSPCs harvested from umbilical cord blood (17% of the total alleles in a population of cells contained both mono- and bi-allele CCR5 disrupted cells) (28). Using these cells, the investigators then showed ZFN treated HSPCs retained the ability to engraft to NSG (NOD/SCID/IL2rγnull) mice and gave rise to polyclonal multi-lineage progeny in which CCR5 was permanently disrupted. They have also shown that mice transplanted with ZFN-modified HSPCs underwent rapid selection for CCR5^{-/-} cells and had significantly lower HIV-1 levels (28). It must be emphasized that ZFN-mediated CCR5 disruption by NHEJ produces a spectrum of CCR5 mutations, of which only a small percentage will result in functional deletion of CCR5 in cells.

Although ZFN mediated gene targeting of the CCR5 locus using donor encoding stem cell factor genes did not yield any homozygous biallelic insertion at the CCR locus of the hiPSCs that we examined, we observed both heterozygous hiPSCs (with a single allele insertion of the pluripotency genes at the CCR5 locus while the second allele remained wild type CCR5) and biallele mutant hiPSCs (with a single allele insertion of the pluripotency genes at the CCR5 locus while the second allele was mutated by NHEJ). Since CRE treatment of the latter hiPSCs results in the disruption of both CCR5 alleles, isolated single CCR5-modified hiPSC colonies could be differentiated into HIV resistant CD34+ hematopoietic stem cells (HSCs) and expanded in vitro for transplantation. A lot of technical challenges still remain for efficient differentiation of hiPSCs into HSCs. However, once these challenges are overcome, ZFN-mediated disruption of CCR5 locus by HR will likely to be the method of choice for generation of HIV-resistant CD34+ cells for autologous transplantation via differentiation of HIV-resistant hiPSC colonies generated from isolated CD34+ primary cells or from cord blood or from adults.

Unlike the Holt et al method, ZFN-mediated approach by HR will generate a uniform biallelic CCR5-disrupted cell population containing a specific mutation from isolated single cell CCR5-modified hiPSC colonies; these could be differentiated into CD34+ hematopoietic stem cells (HSCs) and expanded in vitro for transplantation. HR-driven disruption of CCR5 using targeted insertion of the transgenes results in a specific population of mutant hiPSCs. The properties of a single specific population of CCR5-disrupted cells (either carrying transgenes or just CCR5 mutations) generated from hiPSCs are likely to have quite different properties than a pool of CD34+ cells with different mutations generated by NHEJ. We posit that the hiPSC-derived CD34+ cell population with a defined biallelic CCR5-mutation is likely to more advantageous in transplantation studies as compared to cell population generated by NHEJ. Preliminary experiments using human CD34+ cells from cord blood suggest that targeted addition of four or five pluipotency genes at the CCR5 locus may be necessary for efficient reprogramming of these cells.

### Summary

This approach has enormous potential to substitute biallele CCR5-disrupted single cell colony hiPSCs for CCR5-disrupted T cells and CCR5-disrupted CD34+ cells to provide a functional cure as previously reported, for example by Holt et al., which uses ZFN-mediated disruption of CCR5 by NHEJ to generate a pool of cells, only some of which will be resistant to HIV (28). Thus, this powerful methodology offers an alternative for efficient generation of genetically diverse, patient-specific hiPSCs with disrupted CCR5 loci, both for basic research and for human therapeutics to treat HIV in the future.

References cited herein are listed below for convenience and are hereby incorporated by reference in their entirety.
1. Collins F S (1992) Cystic Fibrosis: Molecular Biology and Therapeutic Implications. Science 256, 774-780.
2. Steen C D (1997). Cystic fibrosis: inheritance, genetics and treatment. Brit J Nursing 6: 192-199.
3. Grubb B R and Boucher R C (1999) Pathophysiology of Gene-Targeted Mouse Models for Cystic Fibrosis. Physiological Reviews 79, S193-S214.
4. Cheng S H, Gregory R J, Marshall J, Paul S, Souza D W, White G A, O'Riordan C and Smith A E (1990). Defective intracellular transport and processing of CFTR is the molecular basis of most cystic fibrosis. Cell 63, 827-834.
5. Grubb B R, Pickles R J, Ye H, Yankaskas J R, Vick R N, et al (1994) Inefficient gene transfer by adenovirus vector to cystic fibrosis airway epithelia of mice and humans. Nature 371:802-806.
6. Johnson L G, Olsen J C, Sarkadi B, Moore K L, Swanstrom R. and Boucher R C (1992) Efficiency of gene transfer for restoration of normal airway epithelial function in cystic fibrosis. Nature Genetics 2, 21-25.
7. Weiss D J (2008) Stem cells and cell therapies for cystic fibrosis and other lung diseases. Pulmonary Pharmacology & Therapeutics 21:588-594.
8. Kim Y-G., Cha J and Chandrasegaran S (1996) Hybrid restriction enzymes: Zinc finger fusions to FokI cleavage domain. Proc. Natl. Acad. Sci USA 93: 1156-1160.
9. Bibikova M, Carroll D, Segal D J, Trautman J K, Smith J, Kim Y-G and Chandrasegaran S (2001) Stimulation of homologous recombination through targeted cleavage by a chimeric nuclease. Molecular and Cellular Biology 21:289-297.
10. Sander J D, Dahlborg E J, Goodwin M J, Cade L, et al (2011) Selection-free zinc-finger-nuclease engineering by context-dependent assembly (CoDA). Nat Methods. 8:67-69.
10a. Ramalingam S, Kandavelou K, Rajenderan R and Chandrasegaran S. (2011). Creating designed zinc finger nucleases with minimal cytotoxicity. J. Mol. Biol. 405: 630-641.
11. Cermak T, Doyle D L, Christian M, Wang L, et al (2011) Efficient design and assembly of custom TALEN and other TAL effector-based constructs for DNA targeting. Nucleic Acids Research 39:e82.
12. Carroll D. (2011) Zinc-finger nucleases: a panoramic view. Curr Gene Ther. 11:2-10.
13. Porteus M H, Baltimore D. (2003) Chimeric nucleases stimulate gene targeting in human cells. Science 300: 763.
14. Urnov F D, Miller J C, Lee Y L, Beausejour C M, Rock J M, et al. (2005) Highly efficient endogenous human gene correction using designed zinc-finger nucleases. Nature 435: 646-51.
15. Kandavelou K, Mani M, Durai S and Chandrasegaran S (2005) ‘Magic’ scissors for genome surgery. Nature Biotechnology 23: 686-687.
16. Wu J, Kandavelou K and Chandrasegaran S (2007) Custom-designed zinc finger nucleases: what is next? Cellular and Molecular Life Sciences 64: 2933-2944.
17. Moehle E A, Rock J M, Lee Y L, Jouvenot Y, et al. (2007) Targeted gene addition into a specified location in the human genome using designed zinc finger nucleases. Proc Natl Acad Sci USA 104, 3055-60.
18. Kandavelou K, Ramalingam S, London V, Mani M, Wu J, Alexeev V, Civin C I and Chandrasegaran S. (2010). Targeted manipulation of mammalian cells using designed zinc finger nucleases. Biochemical Biophysical Research Communications 388: 56-61.
19. Connelly J P, Barker J C, Pruett-Miller S, Porteus M H. (2010) Gene correction by homologous recombination with zinc finger nucleases in primary cells from a mouse model of a generic recessive genetic disease. Mol Ther. 18:1103-1110.
20. Zou J, Maeder M L, Mali P, Pruett-Miller S M, et al (2009) Gene targeting of a disease-related gene in human induced pluripotent stem and embryonic stem cells. Cell Stem Cell 2:97-110.
21. Hockemeyer D, Soldner F, Beard C, Gao Q, Mitalipova M, et al (2009) Efficient targeting of expressed and silent genes in human ESCs and iPSCs using zinc-finger nucleases. Nat Biotechnol. 27:851-857.
22. DeKelver R C, Choi V M, Mohle E A, Pachon D E, et al (2010) Functional genomics, proteomics, regulatory DNA analysis in isogenic settings using zinc finger nuclease-driven transgenesis into a safe harbor locus in the human genome. Genome Research 20: 1133-1142.
23. Hockemeyer D, Wang H, Kiani S, Lai C S, Gao Q, et al (2011) Genetic Engineering of human pluripotent cells using TALE nucleases. (doi:101038/nbt.1927).
24. Zou J, Sweeney C L, Chou B-K, Choi U, et al (2011) Oxidase deficient neutrophils from X-linked chronic granulomatous disease iPS cells: functional correction by zinc finger nuclease mediated safe harbor targeting. (doi:10.1182/blood-2010-12-328161).
25. Bobis-wosowicz S, Osiak A, Rahman S H and Cathomen T (2011) Targeted genome editing in pluripotent stem cells using zinc-finger nucleases. Methods 53:339-346.
26. Perez E E, Wang J, Miller J C, Jouvenot Y, Kim Y A, et al (2008) Establishment of HIV-1 resistance in CD4+ T cells by genome editing using zinc finger nucleases. Nat Biotechnol 26:808-816.
27. Wilen C B, Wang J, Tilton J C, Miller J C, et al (2011) Engineering HIV-Resistant Human CD4+ T Cells with CXCR4-Specific Zinc-Finger Nucleases. PLoS Pathogens 7:e1002020.
28. Holt N, Wang J, Kim K, Friedman G, Wang et at (2010). Human hematopoietic stem/progenitor cells modified by zinc-finger nucleases targeted to CCR5 control HIV-1 in vivo. Nat Biotechnol. 28:839-47.
29. Ye L, Chang J C, Lin C, Qi Z, Yu J and Kan Y W (2010) Generation of induced pluripotent stem cells using site-specific integration with phage integrase. Proc Natl Acad Sci USA 107:19467-19472.
30. Takahashi K, Tanabe K, Ohnuki M, Narita M, Ichisaka T, Tomoda K, Yamanaka S. (2007) Induction of pluripotent stem cells from adult human fibroblasts by defined factors. Cell 131:861-72.
31. Tesson L, Usal C, Ménoret S, Leung E, Niles B J, et al. (2011) Knockout rats generated by embryo microinjection of TALENs. Nature Biotechnology 29:695-696.
32. Sander J D, Cade L, Khayter C, Reyon D, et al. (2011) Targeted gene disruption in somatic zebrafish cells using engineered TALENs. Nature Biotechnology 29:697-698.
33. Huang P, Xiao A, Zhou M, Zhu Z, et al (2011) Heritable gene targeting in zebrafish using customized TALENs. Nature Biotechnology 29:699-700.

1. A method of generating a stem cell from a target somatic cell or primary cell, comprising introducing into the target cell one or more of pluripotency coding sequences at a safe-harbor locus within the cell target genome using site-specific endonucleases, the one or more of a pluripotency coding sequences giving rise upon transcription to a factor that contributes to the reprogramming of said target cell into an induced pluripotent stem cell.

2-3. (canceled)

4. The method of claim 1, wherein the pluripotency coding sequence represents one or more of a gene selected from one or more of from Oct3 or 4 or a factor belonging to the Myc, Klf and Sox families of factors.

5. The method of claim 1, wherein the pluripotency coding sequence represents one or more of a gene selected from one or more of Oct3, Oct4, 1-Myc, n-Myc, c-Myc, Klf1, Klf2, Klf4, Klf15, Sox1, Sox2, Sox3, Sox15 and Sox18.

6. The method of claim 1, wherein the pluripotency coding sequence represents one or more of a gene selected from one or more of Sox2 or Oct4.

7. (canceled)

8. The method of claim 1, wherein the pluripotency coding sequence represents one or more of a gene selected from one or more of Sox2 or Oct4, and is flanked by recombinase recognition sites at the safe-harbor locus.

9. The method of claim 1, wherein the site-specific endonuclease comprises a fusion protein comprising a DNA-binding domain and a FokI cleavage domain or FokI cleavage domain heterodimer variants, wherein the DNA-binding domain binds to a target site in the safe-harbor locus.

10. The method of claim 9, wherein the DNA-binding domain comprises zinc finger protein (ZFP) domain or transcription activator-like effector (TALE) domain.

11. The method of claim 1, wherein the site-specific endonuclease comprises a zinc finger nuclease (ZFN) or a TALE nuclease (TALEN).

12. The method of claim 1, wherein the safe-harbor locus comprises CCR5 or AAVS1.

13. The method of claim 1, wherein the safe-harbor locus is CCR5 or AAVS1, the site-specific endonucleases is zinc finger nucleases (ZFN) or a TALE nucleases (TALEN) which bind to a target site in the CCR5 or AAVS1 gene, and the CCR5 or AAVS1 gene is cleaved, and wherein the pluripotency coding sequences selected from one or more of Sox2 or Oct4, are integrated at the safe-harbor locus.

14-18. (canceled)

19. The method of claim 1, wherein the stem cell comprises non-human embryonic stem cells, non-human adult stem cells, non-human stem progenitor cells or non-human induced pluripotent stem cells.

20. The method of claim 1, comprising the steps of:
a) providing a target somatic cell or primary cell comprising a safe-harbor locus,
b) contacting the cell with a donor construct comprising a reprogramming pluripotency coding sequences and constructs comprising site-specific endonucleases having specificity for a target sequence of interest in the a safe-harbor locus, and
c) culturing the cell to induce reprogramming of the target cell to a stem cell; and
culturing the induced stem cell to remove the reprogramming pluripotency coding sequences from the induced stem cell.

21. The method claim 20, wherein the donor construct is flanked by recombinase recognition site.

22. The method of claim 20, wherein the donor construct is flanked by safe-harbor locus sequence on both the sides for site-specific endonuclease-evoked homology directed repair.

23. The method of claim 20, wherein the safe harbor locus of interest is cleaved and the pluripotency coding sequence is introduced into the genome, thereby giving rise upon transcription to a factor that contributes to the reprogramming of said target cell into an induced pluripotent stem cell.

24. The method claim 1, comprising adding small molecule inhibitor, valproic acid (VPA).

25. The method of claim 1, comprising adding CRE recombinase.

26. The method of claim 1, wherein the stem cell comprises a heterozygous CCR5 single allele mutant resulting homologous recombination (HR).

27-89. (canceled)

90. The method claim 20, comprising adding small molecule inhibitor, valproic acid (VPA).

91. The method of claim 20, comprising adding CRE recombinase


SEQUENCE LISTING (79 sequences)

SEQ ID NO: 1 - DNA, 76 bp, Artificial Sequence
  Description of Artificial Sequence Synthetic oligonucleotide
aggcaggagtcatcctcatcctggttaacataacttcgtatagcatacattatacgaagttatgacattgattatt

SEQ ID NO: 2 - DNA, 76 bp, Artificial Sequence; identical to SEQ ID NO: 1
  Description of Artificial Sequence Synthetic oligonucleotide

SEQ ID NO: 3 - DNA, 76 bp, Artificial Sequence; identical to SEQ ID NO: 1
  Description of Artificial Sequence Synthetic oligonucleotide

SEQ ID NO: 4 - DNA, 76 bp, Artificial Sequence; identical to SEQ ID NO: 1
  Description of Artificial Sequence Synthetic oligonucleotide

SEQ ID NO: 5 - DNA, 76 bp, Artificial Sequence; identical to SEQ ID NO: 1
  Description of Artificial Sequence Synthetic oligonucleotide

SEQ ID NO: 6 - DNA, 76 bp, Artificial Sequence; identical to SEQ ID NO: 1
  Description of Artificial Sequence Synthetic oligonucleotide

SEQ ID NO: 7 - DNA, 76 bp, Artificial Sequence; identical to SEQ ID NO: 1
  Description of Artificial Sequence Synthetic oligonucleotide

SEQ ID NO: 8 - DNA, 76 bp, Artificial Sequence; identical to SEQ ID NO: 1
  Description of Artificial Sequence Synthetic oligonucleotide

SEQ ID NO: 9 - DNA, 72 bp, Artificial Sequence
  Description of Artificial Sequence Synthetic oligonucleotide
ggctctatggataacttcgtatagcatacattatacgaagttatgttaacataaactgcaaaaggccaaacg

SEQ ID NO: 10 - DNA, 72 bp, Artificial Sequence; identical to SEQ ID NO: 9
  Description of Artificial Sequence Synthetic oligonucleotide

SEQ ID NO: 11 - DNA, 72 bp, Artificial Sequence; identical to SEQ ID NO: 9
  Description of Artificial Sequence Synthetic oligonucleotide

SEQ ID NO: 12 - DNA, 72 bp, Artificial Sequence; identical to SEQ ID NO: 9
  Description of Artificial Sequence Synthetic oligonucleotide

SEQ ID NO: 13 - DNA, 72 bp, Artificial Sequence; identical to SEQ ID NO: 9
  Description of Artificial Sequence Synthetic oligonucleotide

SEQ ID NO: 14 - DNA, 72 bp, Artificial Sequence; identical to SEQ ID NO: 9
  Description of Artificial Sequence Synthetic oligonucleotide

SEQ ID NO: 15 - DNA, 72 bp, Artificial Sequence; identical to SEQ ID NO: 9
  Description of Artificial Sequence Synthetic oligonucleotide

SEQ ID NO: 16 - DNA, 72 bp, Artificial Sequence; identical to SEQ ID NO: 9
  Description of Artificial Sequence Synthetic oligonucleotide

SEQ ID NO: 17 - DNA, 61 bp, Artificial Sequence
  Description of Artificial Sequence Synthetic oligonucleotide
gggcaacatgctggtcatcctcatccaactgcaaaaggctgaagagcatgactgacatcta

SEQ ID NO: 18 - DNA, 66 bp, Artificial Sequence
  Description of Artificial Sequence Synthetic oligonucleotide
gggcaacatgctggtcatcctcatcctgataaaaactgcaaaaggctgaagagcatgactgacatc

SEQ ID NO: 19 - DNA, 58 bp, Artificial Sequence
  Description of Artificial Sequence Synthetic oligonucleotide
gggcaacatgctggtcatcctcatactgcaaaaggctgaagagcatgactgacatcta

SEQ ID NO: 20 - DNA, 63 bp, Artificial Sequence
  Description of Artificial Sequence Synthetic oligonucleotide
gggcaacatgctggtcatcctcatcctaaactgcaaaaggctgaagagcatgactgacatcta

SEQ ID NO: 21 - DNA, 75 bp, Artificial Sequence
  Description of Artificial Sequence Synthetic oligonucleotide
gtcatcctcatcctggttaacataacttcgtatagcatacattatacgaagttatgttaacataaactgcaaaag

SEQ ID NO: 22 - DNA, 66 bp, Artificial Sequence
  Description of Artificial Sequence Synthetic oligonucleotide
tggttttgtgggcaacatgctggtcatcctcatcctgataaactgcaaaaggctgaagagcatgac

SEQ ID NO: 23 - DNA, 75 bp, Artificial Sequence; identical to SEQ ID NO: 21
  Description of Artificial Sequence Synthetic oligonucleotide

SEQ ID NO: 24 - DNA, 66 bp, Artificial Sequence; identical to SEQ ID NO: 22
  Description of Artificial Sequence Synthetic oligonucleotide

SEQ ID NO: 25 - DNA, 74 bp, Artificial Sequence
  Description of Artificial Sequence Synthetic oligonucleotide
gtcatcctcatcctggttaacataacttcgtatagcatacattatacgaagttatgttaacataactgcaaaag

SEQ ID NO: 26 - DNA, 66 bp, Artificial Sequence; identical to SEQ ID NO: 22
  Description of Artificial Sequence Synthetic oligonucleotide

SEQ ID NO: 27 - DNA, 75 bp, Artificial Sequence; identical to SEQ ID NO: 21
  Description of Artificial Sequence Synthetic oligonucleotide

SEQ ID NO: 28 - DNA, 66 bp, Artificial Sequence; identical to SEQ ID NO: 22
  Description of Artificial Sequence Synthetic oligonucleotide

SEQ ID NO: 29 - DNA, 75 bp, Artificial Sequence; identical to SEQ ID NO: 21
  Description of Artificial Sequence Synthetic oligonucleotide

SEQ ID NO: 30 - DNA, 61 bp, Artificial Sequence; identical to SEQ ID NO: 17
  Description of Artificial Sequence Synthetic oligonucleotide

SEQ ID NO: 31 - DNA, 75 bp, Artificial Sequence; identical to SEQ ID NO: 21
  Description of Artificial Sequence Synthetic oligonucleotide

SEQ ID NO: 32 - DNA, 61 bp, Artificial Sequence; identical to SEQ ID NO: 17
  Description of Artificial Sequence Synthetic oligonucleotide

SEQ ID NO: 33 - DNA, 75 bp, Artificial Sequence; identical to SEQ ID NO: 21
  Description of Artificial Sequence Synthetic oligonucleotide

SEQ ID NO: 34 - DNA, 66 bp, Artificial Sequence; identical to SEQ ID NO: 18
  Description of Artificial Sequence Synthetic oligonucleotide

SEQ ID NO: 35 - DNA, 75 bp, Artificial Sequence; identical to SEQ ID NO: 21
  Description of Artificial Sequence Synthetic oligonucleotide

SEQ ID NO: 36 - DNA, 66 bp, Artificial Sequence; identical to SEQ ID NO: 18
  Description of Artificial Sequence Synthetic oligonucleotide

SEQ ID NO: 37 - DNA, 66 bp, Artificial Sequence
  Description of Artificial Sequence Synthetic oligonucleotide
gggcaacatgctggtcatcctcatcctgataaactgcaaaaggctgaagagcatgactgacatcta

SEQ ID NO: 38 - DNA, 59 bp, Artificial Sequence
  Description of Artificial Sequence Synthetic oligonucleotide
gggcaacatgctggtcatcctcatcactgcaaaaggctgaagagcatgactgacatcta

SEQ ID NO: 39 - DNA, 66 bp, Artificial Sequence; identical to SEQ ID NO: 37
  Description of Artificial Sequence Synthetic oligonucleotide

SEQ ID NO: 40 - DNA, 65 bp, Artificial Sequence
  Description of Artificial Sequence Synthetic oligonucleotide
gggcaacatgctggtcatcctcatctgataaactgcaaaaggctgaagagcatgactgacatcta

SEQ ID NO: 41 - DNA, 44 bp, Artificial Sequence
  Description of Artificial Sequence Synthetic oligonucleotide
gggcaacatgctggtcatcctctgaagagcatgactgacatcta

SEQ ID NO: 42 - DNA, 70 bp, Artificial Sequence
  Description of Artificial Sequence Synthetic oligonucleotide
aggcaggagtcatcctcatcctgcaattggacattgattattgactagttaaccatgcagaggtcgcctc

SEQ ID NO: 43 - DNA, 70 bp, Artificial Sequence; identical to SEQ ID NO: 42
  Description of Artificial Sequence Synthetic oligonucleotide

SEQ ID NO: 44 - DNA, 70 bp, Artificial Sequence; identical to SEQ ID NO: 42
  Description of Artificial Sequence Synthetic oligonucleotide

SEQ ID NO: 45 - DNA, 70 bp, Artificial Sequence; identical to SEQ ID NO: 42
  Description of Artificial Sequence Synthetic oligonucleotide

SEQ ID NO: 46 - DNA, 70 bp, Artificial Sequence; identical to SEQ ID NO: 42
  Description of Artificial Sequence Synthetic oligonucleotide

SEQ ID NO: 47 - DNA, 69 bp, Artificial Sequence
  Description of Artificial Sequence Synthetic oligonucleotide
gcagtacgattccatccagaactgtgccttctagttgccagcaattgataaactgcaaaaggccaaacg

SEQ ID NO: 48 - DNA, 69 bp, Artificial Sequence; identical to SEQ ID NO: 47
  Description of Artificial Sequence Synthetic oligonucleotide

SEQ ID NO: 49 - DNA, 69 bp, Artificial Sequence; identical to SEQ ID NO: 47
  Description of Artificial Sequence Synthetic oligonucleotide

SEQ ID NO: 50 - DNA, 69 bp, Artificial Sequence; identical to SEQ ID NO: 47
  Description of Artificial Sequence Synthetic oligonucleotide

SEQ ID NO: 51 - DNA, 69 bp, Artificial Sequence; identical to SEQ ID NO: 47
  Description of Artificial Sequence Synthetic oligonucleotide

SEQ ID NO: 52 - DNA, 30 bp, Artificial Sequence
  Description of Artificial Sequence Synthetic primer
caactcaaactacaaacacaaacttcacag

SEQ ID NO: 53 - DNA, 25 bp, Artificial Sequence
  Description of Artificial Sequence Synthetic primer
ggaaagtcccgttgattttggtgcc

SEQ ID NO: 54 - DNA, 21 bp, Artificial Sequence
  Description of Artificial Sequence Synthetic primer
ctgtgccttctagttgccagc

SEQ ID NO: 55 - DNA, 26 bp, Artificial Sequence
  Description of Artificial Sequence Synthetic primer
gagtttgatgcttattgaatgtgtag

SEQ ID NO: 56 - DNA, 24 bp, Artificial Sequence
  Description of Artificial Sequence Synthetic primer
atggattatcaagtgtcaagtcca

SEQ ID NO: 57 - DNA, 22 bp, Artificial Sequence
  Description of Artificial Sequence Synthetic primer
tcacaagcccacagatatttcc

SEQ ID NO: 58 - DNA, 28 bp, Artificial Sequence
  Description of Artificial Sequence Synthetic primer
gttgccctaaggattaaatgaatgaatg

SEQ ID NO: 59 - DNA, 28 bp, Artificial Sequence
  Description of Artificial Sequence Synthetic primer
ctccatgcgcaccttgaagcgcatgaac

SEQ ID NO: 60 - DNA, 28 bp, Artificial Sequence
  Description of Artificial Sequence Synthetic primer
gcagtacgattccatccagaaactgctg

SEQ ID NO: 61 - DNA, 27 bp, Artificial Sequence
  Description of Artificial Sequence Synthetic primer
ggatgaatcttagaccctctataacag

SEQ ID NO: 62 - DNA, 24 bp, Artificial Sequence
  Description of Artificial Sequence Synthetic primer
acaagattttatttggtgagatgg

SEQ ID NO: 63 - DNA, 22 bp, Artificial Sequence
  Description of Artificial Sequence Synthetic primer
agaattgatactgactgtatgg

SEQ ID NO: 64 - protein, 196 aa, Artificial Sequence
  Description of Artificial Sequence Synthetic polypeptide
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQAREMERYVEENQTRNKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 65 - protein, 196 aa, Artificial Sequence
  Description of Artificial Sequence Synthetic polypeptide
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQDYVKENQTRNKHINPNEWWKVYPSSVTEFKFLFVSGHFKGNYKAQLTRLNHKTNCNGAVLSVEELLIGGEMIKAGTLTLEEVRRKFNNGEINF

SEQ ID NO: 66 - DNA, 21 bp, Artificial Sequence
  Description of Artificial Sequence Synthetic oligonucleotide
gaacgcggaacgctggcccgc

SEQ ID NO: 67 - protein, 7 aa, Artificial Sequence
  Description of Artificial Sequence Synthetic peptide
ERGTLAR

SEQ ID NO: 68 - DNA, 21 bp, Artificial Sequence
  Description of Artificial Sequence Synthetic oligonucleotide
gaccgctcggacttgacgcgc

SEQ ID NO: 69 - protein, 7 aa, Artificial Sequence
  Description of Artificial Sequence Synthetic peptide
DRSDLTR

SEQ ID NO: 70 - DNA, 21 bp, Artificial Sequence
  Description of Artificial Sequence Synthetic oligonucleotide
caatcctctgacttgacgcgc

SEQ ID NO: 71 - protein, 7 aa, Artificial Sequence
  Description of Artificial Sequence Synthetic peptide
QSSDLTR

SEQ ID NO: 72 - DNA, 21 bp, Artificial Sequence
  Description of Artificial Sequence Synthetic oligonucleotide
gacagatccaaccttacccgc

SEQ ID NO: 73 - protein, 7 aa, Artificial Sequence
  Description of Artificial Sequence Synthetic peptide
DRSNLTR

SEQ ID NO: 74 - protein, 7 aa, Artificial Sequence
  Description of Artificial Sequence Synthetic peptide
RSDHLTK

SEQ ID NO: 75 - DNA, 21 bp, Artificial Sequence
  Description of Artificial Sequence Synthetic oligonucleotide
caatcctctaatctcgctcgc

SEQ ID NO: 76 - protein, 7 aa, Artificial Sequence
  Description of Artificial Sequence Synthetic peptide
QSSNLAR

SEQ ID NO: 77 - DNA, 10 bp, Homo sapiens
gctgccgccc

SEQ ID NO: 78 - DNA, 10 bp, Homo sapiens
gaaggggaca

SEQ ID NO: 79 - DNA, 21 bp, Artificial Sequence
  Description of Artificial Sequence Synthetic oligonucleotide
cgcagcgatcatctcaccaaa